(19) United States
(12) Patent Application Publication
(10) Pub. No.: US 2004/0242559 A1
(43) Pub. Date: Dec. 2, 2004

(54) Novel indole derivatives, preparation thereof as medicinal products and pharmaceutical compositions, and especially as KDR inhibitors

(75) Inventors: Antonio Ugolini, Massy (FR); Herve Bouchard, Thiais (FR)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 10/830,826

(22) Filed: Apr. 23, 2004

Related U.S. Application Data
(60) Provisional application No. 60/485,785, filed on Jul. 8, 2003.

(30) Foreign Application Priority Data
Apr. 25, 2003 (FR) 0305088

(51) Int. Cl. A61K 31/541; A61K 31/5377; A61K 31/496; A61K 31/416; C 07D 4 3/14

(52) U.S. Cl. 514/210.21; 514/227.5; 514/233.5; 514/254.06; 514/406; 544/60; 544/140; 544/371; 548/364.7

(57) ABSTRACT

The invention relates to compounds of formula (I):

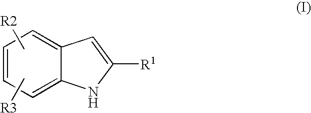

in which R1 represents pyrazolyl or indazolyl, R2 and R3 especially represent H, halogen, hydroxyl, nitro, cyano, R4, —OR4, —COR4, —OC(═O)R4, —C(═O)OR4, —C(═O)OH, —N(R5)C(═O)R4, —N(R5)C(═O)OR4, —S(O)nR4, —S(O)nOR4, —N(R5)SO2R4, —NY1Y2, —C(═O)NY1Y2, —N(R5)C(═O)NY1Y2, —S(O)nNY1Y2 and —OC(═O)NY1Y2, R4 especially represents alkyl, alkenyl, cycloalkyl, aryl, heteroaryl and heterocycloalkyl, R5 especially represents H, alkyl, alkenyl, cycloalkyl, heterocycloalkyl, aryl and heteroaryl, Y1 and Y2 especially represent H, alkyl, alkenyl, cycloalkyl, heterocycloalkyl, aryl, arylcarboxyl, heteroaryl and heteroarylcarboxy, which are optionally substituted, or Y1 and Y2 form with N an amino ring, n represents 0 to 2, all these radicals being optionally substituted, these products being in all the isomeric forms and the salts, as medicinal products, especially as KDR inhibitors.

### CROSS-REFERENCE TO RELATED APPLICATIONS

This application claims priority of French Patent Application 0305088, filed Apr. 25, 2003, and claims the benefit of U.S. Provisional Applcation 60/485,785 filed Jul. 8, 2003.

### BACKGROUND OF THE INVENTION

The present invention relates to novel indole derivatives, to a process for preparing them, to the novel intermediates obtained, to their use as medicinal products, to pharmaceutical compositions containing them and to the novel use of such indole derivatives.

### SUMMARY OF THE INVENTION

One subject of the invention is thus novel indole derivatives endowed with inhibitory effects on protein kinases.

The products of the present invention may thus be used especially for preventing or treating complaints that may be modified by inhibiting the activity of protein kinases.

The products of the present patent application as protein kinase inhibitors may thus be used for treating or preventing diseases chosen from the following group: cancer, atherosclerosis, muscular degeneration diseases, obesity, Parkinson's disease, depression, schizophrenia, cranial trauma, spinal cord injury, Alzheimer's disease, neuropathic pain syndrome, amyotrophic lateral sclerosis, cachexia, osteoporosis and various fibrotic disorders.

The products of the present patent application as protein kinase inhibitors may be used most particularly for treating or preventing cancers, especially including breast cancer, colon cancer, lung cancer and prostate cancer.

Cancer remains a disease for which the existing treatments are insufficient. Certain protein kinases play an important role in many cancers. The inhibition of such protein kinases is potentially important in cancer chemotherapy, especially for stopping the growth or survival of tumours.

The present invention thus relates to the identification of novel products that inhibit such protein kinases.

The inhibition and regulation of protein kinases is an especially powerful new mechanism of action for treating a large number of solid tumours.

Such afflictions that may be treated by the products of the present application are thus most particularly solid tumours.

### DETAILED DESCRIPTION

Such protein kinases belong especially to the following group: EGFR, Fak, FLK-1, FGFR1, FGFR2, FGFR3, FGFR4, FGFR5, flt-1, IGF-1R, KDR, PLK,PDGFR, tie2, VEGFR, AKT, Raf and Aurora 1 or 2.

Protein Kinases

Protein kinases are a family of enzymes that catalyse the phosphorylation of hydroxyl groups of specific residues of proteins, such as tyrosine, serine or threonine residues. Such phosphorylations may greatly modify the function of the proteins; thus, protein kinases play an important role in regulating a wide variety of cell processes including, especially, metabolism, cell proliferation, cell differentiation or cell survival. Among the various cellular functions in which the activity of a protein kinase is involved, certain processes represent attractive targets for treating certain diseases. As an example, mention may be made especially of angiogenesis and the control of the cell cycle, in which protein kinases can play an essential role. These processes are essential for the growth of solid tumours and also for other diseases.

Protein kinases participate in signalling events that control the activation, growth and differentiation of cells in response either to extracellular mediators or to changes in the environment. In general, these kinases belong to two groups: those that preferentially phosphorylate serine and/or threonine residues and those that preferentially phosphorylate tyrosine residues [S. K. Hanks and T. Hunter, FASEB. J., 1995, 9, pages 576-596]. Serine/threonine kinases are, for example, the isoforms of protein kinases C [A. C. Newton, J. Biol. Chem., 1995, 270, pages 28495-28498] and a group of cycline-dependent kinases, for instance cdc2 [J. Pines, Trends in Biochemical Sciences, 1995, 18, pages 195-197]. Tyrosine kinases comprise growth factor receptors, for instance the epidermal growth factor (EGF) receptor [S. Iwashita and M. Kobayashi, Cellular Signalling, 1992, 4, pages 123-132], and cytosolic kinases, for instance p56tck, p59fYn and ZAP-70 and the csk kinases [C. Chan et. al., Ann. Rev. Immunol., 1994, 12, pages 555-592]. Abnormally high levels of protein kinase activity have been implicated in many diseases, resulting from abnormal cellular functions. This may arise, either directly or indirectly, from a dysfunction in the mechanisms for controlling kinase activity, associated for example with a mutation, an overexpression or an inappropriate activation of the enzyme, or with an overproduction or underproduction of cytokines or growth factors, which are also involved in signal transduction upstream or downstream of the kinases. In all these cases, a selective inhibition of the action of kinases suggests a potential beneficial effect.

Among these protein kinases, mention is made more particularly of the protein kinases KDR, Fak, tie2, Aurora, AKT and IGF-1R,

Mention is made more particularly of the protein kinase KDR.

Mention is also made of the protein kinase FAK.

Mention is also made of the protein kinase Tie-2.

Mention is also made of the protein kinase Aurora.

Mention is also made of the protein kinase AKT.

Mention is also made of the protein kinase IGF1-R.

KDR

Angiogenesis is the process in which new vessels are formed from already-existing vessels. Should the need arise, the vascular system has the potential to generate a network of new vessels so as to maintain the correct functioning of the tissues and organs.

Angiogenesis is a complex multi-step process involving activation, migration, proliferation and survival of endothelial cells.

In adults, angiogenesis is fairly limited, appearing mainly only in the processes of repair after an injury or of vascularization of the endometrium. (Merenmies et al., Cell Growth & Differentiation, 8, 3-10, 1997). However, uncontrolled angiogenesis is found in certain pathologies such as retinopathy, psoriasis, rheumatoid arthritis, diabetes, muscle degeneration or cancer (solid tumours) (Folkman, Nature Med., 1, 27-31, 1995). The kinase proteins whose involvement it has been possible to demonstrate in the angiogenesis process include three members of the family of growth factor receptor tyrosine kinases: VEGF-R2 (vascular endothelial growth factor receptor 2, also known as KDR, kinase insert domain receptor, or FLK-1), FGF-R (fibroblast growth factor receptor) and TEK (also known as Tie-2).

In conjunction with other systems, the Vascular Endothelial Growth Factor receptors (VEGFRs) transmit signals involved in the migration, proliferation and survival of endothelial cells. The family VEGFR includes VEGFR-1 (Flt-1), VEGFR-2 (KDR) and VEGFR3 (Flt4).

The receptor VEGF-R2 (KDR), which is expressed only in the endothelial cells, binds to the angiogenic growth factor VEGF, and thus serves as a transduction signal mediator via the activation of its intracellular kinase domain. Thus, the direct inhibition of the kinase activity of VEGF-R2 makes it possible to reduce the phenomenon of angiogenesis in the presence of exogenous VEGF (Strawn et al., Cancer Research, 56, 3540-3545, 1996), this process being demonstrated especially with the aid of VEGF-R2 mutants (Millauer et al., Cancer Research, 56, 1615-1620, 1996). The VEGFR-2 receptor appears to have no other function in adults than that associated with the angiogenic activity of VEGF. Thus, a selective inhibitor of the kinase activity of VEGF-R2 should show only little toxicity.

In addition to this central role in the dynamic angiogenic process, recent results suggest that the expression of VEGF contributes towards the survival of tumoral cells after chemotherapy and radiotherapy, underlining the potential synergy of KDR inhibitors with other agents (Lee C. G., Heijn M. et al., (2000), Cancer Research, 60 (19), 5565-70).

The KDR inhibitors thus especially constitute anti-angiogenic agents.

Angiogenesis inhibitors might thus be used as a first line treatment against the emergence or regrowth of malignant tumours.

The inhibition or regulation of VEGFR-2 (KDR) thus provides a powerful new mechanism of action for the treatment of a large number of solid tumours.

FAK

Among the kinases for which modulation of activity is desired, FAK (Focal Adhesion Kinase) is also a preferred kinase.

FAK is a cytoplasmic tyrosine kinase that plays an important role in transducing the signal transmitted by the integrins, a family of heterodimeric cellular adhesion receptors. FAK and the integrins are colocated in perimembrane structures known as adhesion plaques. It has been shown in many cell types that the activation of FAK and its phosphorylation on tyrosine residues and in particular its autophosphorylation on tyrosine 397 were dependent on the binding of integrins to their extracellular ligands and thus induced during cellular adhesion [Kornberg L, et al. J. Biol. Chem. 267(33): 23439-442 (1992)]. Autophosphorylation on tyrosine 397 of FAK represents a binding site for another tyrosine kinase, Src, via its SH2 domain [Schaller et al. Mol. Cell. Biol. 14: 1680-1688 1994; Xing et al. Mol. Cell. Biol. 5: 413-421 1994]. Src may then phosphorylate FAK on tyrosine 925, thus recruiting the adapter protein Grb2 and inducing in certain cells activation of the ras and MAP kinase pathway involved in controlling cell proliferation [Schlaepfer et al. Nature; 372: 786-791 1994; Schlaepfer et al. Prog. Biophy. Mol. Biol. 71: 435-478 1999; Schlaepfer and Hunter, J. Biol. Chem. 272: 13189-13195 1997].

The activation of FAK may also induce the jun NH2-terminal kinase (JNK) signalling pathway and result in the progression of cells towards the G1 phase of the cell cycle [Oktay et al., J. Cell. Biol. 145: 1461-1469 1999]. Phosphatidylinositol-3-OH kinase (PI3-kinase) also binds to FAK on tyrosine 397 and this interaction might be necessary for activating PI3-kinase [Chen and Guan, Proc. Nat. Acad. Sci. USA. 91: 10148-10152 1994; Ling et al. J. Cell. Biochem. 73: 533-544 1999]. The FAK/Src complex phosphorylates various substrates, for instance paxillin and p130CAS in fibroblasts [Vuori et al. Mol. Cell. Biol. 16: 2606-2613 1996].

The results of numerous studies support the hypothesis that FAK inhibitors might be useful in treating cancer. Studies have suggested that FAK might play an important role in cell proliferation and/or survival in vitro. For example, in CHO cells, some authors have demonstrated that the overexpression of pl25FAK leads to an acceleration of the transition G1 to S, suggesting that pl25FAK promotes cell proliferation [Zhao J.-H et al. J. Cell Biol. 143: 1997-2008 1998]. Other authors have shown that tumour cells treated with FAK antisense oligonucleotides lose their adhesion and enter into apoptosis (Xu et al., Cell Growth Differ. 4: 413-418 1996). It has also been demonstrated that FAK promotes the migration of cells in vitro. Thus, fibroblasts that are deficient for FAK expression (FAK “knockout” mice) show a rounded morphology and deficiencies in cellular migration in response to chemotactic signals, and these defects are eliminated by re-expression of FAK [D J. Sieg et al., J. Cell Science. 112: 2677-91 1999]. Overexpression of the C-terminal domain of FAK (FRNK) blocks the stretching of adherent cells and reduces cell migration in vitro [Richardson A. and Parsons J. T. Nature. 380: 538-540 1996]. Overexpression of FAK in CHO or COS cells or in human astrocytoma cells promotes migration of the cells. The involvement of FAK in promotion of the proliferation and migration of cells in many cell types in vitro suggests the potential role of FAK in neoplastic processes. A recent study has effectively demonstrated the increase in the proliferation of tumour cells in vivo after inducing the expression of FAK in human astrocytoma cells [Cary L. A. et al. J. Cell Sci. 109: 1787-94 1996; Wang D et al. J. Cell Sci. 113: 4221-4230 2000]. Furthermore, immunohistochemical studies of human biopsies have demonstrated that FAK was overexpressed in prostate cancer, breast cancer, thyroid cancer, colon cancer, melanoma, brain cancer and lung cancer, the level of expression of FAK being directly correlated to the tumours showing the most aggressive phenotype [Weiner T M, et al. Lancet. 342 (8878): 1024-1025 1993; Owens et al. Cancer Research. 55: 2752-2755 1995; Maung K. et al. Oncogene 18: 6824-6828 1999; Wang D et al. J. Cell Sci. 113: 4221-4230 2000).

Tie-2

Tie-2 (TEK) is a member of a family of tyrosine kinase receptors, which is specific to endothelial cells. Tie2 is the first receptor with tyrosine kinase activity for which both the agonist (angiopoietin 1 or Ang1) which stimulates the autophosphorylation of the receptor and cell signalling [S. Davis et al. (1996) Cell 87, 1161-1169] and the antagonist (angiopoietin 2 or Ang2) [P. C. Maisonpierre et al. (1997) Science 277, 55-60] are known. Angiopoietin 1 can synergize with VEGF in the final stages of neoangiogenesis [AsaharaT. Circ. Res. (1998) 233-240]. Knock-out experiments and transgenic manipulations of the expression of Tie2 or of Ang1 lead to animals that present vascularization defects [D. J. Dumont et al. (1994) Genes Dev. 8, 1897-1909 and C. Suri (1996) Cell 87, 1171-1180]. The binding of Ang1 to its receptor leads to autophosphorylation of the kinase domain of Tie2, which is essential for neovascularization and also for the recruitment and interaction of blood vessels with the pericytes and smooth muscle cells; these phenomena contribute towards the maturation and stability of the newly formed blood vessels [P. C. Maisonpierre et al. (1997) Science 277, 55-60]. Lin et al. (1997) J. Clin. Invest. 100, 8: 2072-2078 and Lin P. (1998) PNAS 95, 8829-8834 have shown an inhibition of tumour growth and vascularization, and also a reduction in lung metastases, during adenoviral infections or injections of the extracellular domain of Tie-2 (Tek) into models of melanoma and breast tumour xenografts.

Tie2 inhibitors may be used in situations in which neovascularization takes place inappropriately (i.e. in diabetic retinopathy, chronic inflammation, psoriasis, Kaposi's sarcoma, chronic neovascularization due to macular degeneration, rheumatoid arthritis, infantile haemoangioma and cancer.

Aurora 2

Many proteins involved in chromosomal segregation and spindle assembly have been identified in yeast and drosophila. Disorganization of these proteins results in non-segregation of the chromosomes and in monopolar or disorganized spindles. Among these proteins, certain kinases, including Aurora and Ipl1, originating from S. cerevisiae and drosophila, respectively, are necessary for segregation of the chromosomes and separation of the centrosome. A human analogue of yeast Ipl1 has recently been cloned and characterized by different laboratories. This kinase, known as aurora2, STK15 or BTAK, belongs to the serine/threonine kinase family. Bischoff et al. have shown that Aurora2 is oncogenic, and is amplified in human colorectal cancers (EMBO J, 1998, 17, 3052-3065). This has also been exemplified in cancers involving epithelial tumours, such as breast cancer.

AKT

The protein kinase AKT (also known as PKB) and phosphoinositide 3-kinase (PI3K) are involved in a cell signaling pathway that transmits signals from membrane receptor-activating growth factors.

This transduction pathway is involved in many cell functions: regulation of apoptosis, control of transcription and translation, glucose metabolism, angiogenesis and mitochondrial integrity. The serine/threonine kinase AKT, which was first identified as an important agent in the insulin-dependent signaling pathways that regulate metabolic responses, was then identified as a mediator playing a key role in survival induced by growth factors. It has been shown that AKT can inhibit death by apoptosis induced by various stimuli, in a certain number of cell types and of tumour cells. In accordance with these findings, it has been shown that AKT can, via phosphorylation of given serine residues, inactivate BAD, GSK3β, caspase-9 and Forkhead transcription factor, and activate IKKalpha and e-NOS. It is interesting to note that the protein BAD is found hyperphosphorylated in 11 human tumour cell lines out of 41 studied. Furthermore, it has been shown that hypoxia modulates the induction of VEGF in Ha-ras-transformed cells by activating the PI3K/AKT pathway and by involving the sequence of binding of the transcription factor HIF-1 (hypoxia inducible factor-1) known as HRE for “hypoxia-responsive-element”.

AKT plays a very important role in cancer pathologies. The amplification and/or overexpression of AKT has been reported in many human tumours, for instance gastric carcinoma (amplification of AKT1), ovarian, breast or pancreatic carcinomas (amplification and overexpression of AKT2) and breast carcinomas deficient in oestrogen receptors, and also androgen-independent prostate carcinomas (overexpression of AKT3). Furthermore, AKT is constitutively activated in all PTEN (−/−) tumours, the phosphatase PTEN being deleted or inactivated via mutations in many types of tumours, for instance ovarian, prostate and endometrial carcinomas, glioblastomas and melanomas. AKT is also involved in the oncogenic activation of bcr-abl (references: Khawaja A., Nature 1999, 401, 33-34; Cardone et al. Nature 1998, 282, 1318-1321; Kitada S. et al., Am J Pathol 1998 Jan; 152(1): 51-61; Mazure N M et al. Blood 1997, 90, 3322-3331; Zhong H. et al. Cancer Res. 2000, 60, 1541-1545).

IGF-I-R (Insulin Growth Factor-1 Receptor)

The type-1 receptor for the insulin-like growth factor (IGF-I-R) is a transmembrane receptor with tyrosine kinase activity which binds firstly to IGFI, but also to IGFII and to insulin with lower affinity. The binding of IGF1 to its receptor results in oligomerization of the receptor, the activation of tyrosine kinase and the intermolecular autophosphorylation and phosphorylation of cell substrates (main substrates: IRS1 and Shc). The receptor activated by its ligand induces mitogenic activity in normal cells. However, IGF-I-R plays an important role in “abnormal” growth.

Several clinical reports underline the important role of the IGF-I pathway in the development of human cancers:

IGF-I-R is often found overexpressed in many tumour types (breast, colon, lung, sarcoma, etc.) and its presence is often associated with a more aggressive phenotype.

High concentrations of circulating IGF1 are strongly correlated to a risk of prostate, lung and breast cancer.

Furthermore, it has been widely documented that IGF-I-R is necessary for establishing and maintaining the transformed phenotype both in vitro and in vivo [Baserga R, Exp. Cell. Res., 1999, 253, pages 1-6]. The kinase activity of IGF-I-R is essential to the transformation activity of several oncogenes: EGFR, PDGFR, the major T antigen of the SV40 virus, activated Ras, Raf, and v-Src. The expression d'IGF-I-R in normal fibroblasts induces a neoplastic phenotype, which can then result in tumour formation in vivo. The expression of IGF-I-R plays an important role in substrate-independent growth. IGF-I-R has also been shown to be a protector in chemotherapy-induced and radiation-induced apoptosis and cytokine-induced apoptosis. Furthermore, the inhibition of endogenous IGF-I-R with a negative dominant, the formation of a triple helix or the expression of an antisense nucleic acid results in suppression of the in vitro transforming activity and a reduction in tumour growth in animal models.

The present patent application thus relates particularly to novel VEGFR-2 (KDR) receptor inhibitors that may be used especially for anti-angiogenic treatment in oncology.

The present invention also relates to novel FAK receptor inhibitors that may be used for treatments in oncology. The present invention also relates to novel Tie-2 receptor inhibitors that may be used for treatments in oncology.

The present invention also relates to novel Aurora receptor inhibitors that may be used for treatments in oncology.

The present invention also relates to novel AKT receptor inhibitors that may be used for treatments in oncology. The present invention thus also relates to novel IGF-1R receptor inhibitors that may be used for treatments in oncology.

One subject of the present invention is thus the products of formula (I):

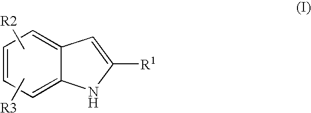

in which:

R1 represents a pyrazolyl or indazolyl radical, these pyrazolyl or indazolyl radicals optionally being substituted with one or more radicals chosen from halogen atoms and hydroxyl, nitro, cyano, R4, OR4, SR4, —COR4, —OC(═O)R4, —C(═O)OR4, free or salified —C(═O)OH, —N(R5)C(═O)R4, —N(R5)C(═O)OR4, —S(O)nR4, —S(O)nOR4, —N(R5)SO2R4, —OS(O)nR4, —NY1Y2, —C(═O)NY1Y2, —OC(═O)NY1Y2, —N(R5)C(═O)NY1Y2, —S(O)nNY1Y2 and thienyl radicals, which are optionally substituted,

R2 and R3 are such that:

either R2 and R3, which may be identical or different, are chosen from a hydrogen atom, halogen atoms and hydroxyl, nitro, cyano, R4, —OR4, —COR4, —OC(═O)R4, —C(═O)OR4, —C(═O)OH, —N(R5)C(═O)R4, —N(R5)C(═O)OR4, —S(O)nR4, —S(O)nOR4, —N(R5)SO2R4, —NY1Y2, —C(═O)NY1Y2, —N(R5)C(═O)NY1Y2, —S(O)nNY1Y2 and —OC(═O)NY1Y2 radicals,

or R2 and R3 form, with the phenyl residue of the indole radical, a 4- to 6-membered carbon-based ring optionally containing one or more identical or different hetero atoms chosen from O, N and S, this ring optionally being substituted,

R4 represents alkyl, alk-NY1Y2, alk-CO—NY1Y2, alkenyl, alkynyl, cycloalkyl, aryl, heteroaryl, cycloalkylalkyl, heterocycloalkyl, heteroarylalkyl and arylalkyl, all these radicals optionally being substituted,

R5 represents hydrogen, alkyl, alkenyl, cycloalkyl, heterocycloalkyl, aryl, heteroaryl, arylalkyl, cycloalkylalkyl, heteroarylalkyl and heterocycloalkylalkyl which are optionally substituted,

Y1 and Y2 are such that:

either Y1 and Y2, which may be identical or different, represent H, alkyl, alkenyl, cycloalkyl, heterocycloalkyl, heterocycloalkylalkyl, aryl, arylalkyl, arylcarboxyl, heteroaryl, heteroarylalkyl and heteroarylcarboxyl, all these radicals being optionally substituted,

or Y1 and Y2 form, together with the nitrogen atom to which they are attached, an optionally substituted cyclic amino radical,

all the alkyl, alkenyl, alkynyl, cycloalkyl, cycloalkylalkyl, heterocycloalkyl, heterocycloalkylalkyl, aryl, aryloxy, arylalkyl, arylcarboxyl, heteroaryl, heteroarylalkyl and heteroarylcarboxyl radicals being optionally substituted with one or more radicals chosen from halogen atoms and hydroxyl, alkoxy, alkyl, hydroxyalkyl, carboxyalkyl, cyano, nitro, trifluoromethyl, trifluoromethoxy radicals, a carboxyl radical that is free, salified or esterified with an optionally substituted alkyl radical, —Nalk-COalk, —NH—COalk, S(O)n-alk, NH—S(O)n-alkyl, —NHCO—NY3Y4, —C(═O)—NY3Y4 and S(O)n-NY3Y4, aryl, arylalkoxy, aryloxy, aryloxyalkyl, heteroaryl and heterocycloalkyl radicals, which are optionally substituted,

with Y3 and Y4, which may be identical or different, representing hydrogen, alkyl or aryl, which are optionally substituted,

these latter alkyl (alk), heterocycloalkyl, aryl and heteroaryl radicals themselves optionally being substituted with one or more radicals chosen from halogen atoms and alkyl, free, salified or esterified carboxyl, amino, alkylamino, dialkylamino and phenylamino, hydroxyl, alkoxy and NHCO alkyl radicals,

all the phenyl radicals also being optionally substituted with a dioxole radical,

n represents an integer from 0 to 2,

alk represents alkyl of 1 to 6 carbon atoms,

said compounds of formula (I) being in all the possible racemic, enantiomeric and diastereoisomeric isomer forms, and also the addition salts with mineral and organic acids or with mineral bases.

Preferably, the products of formula (I) of the present invention do not represent:

i) products of formula (I) in which R2 and R3 both represent a nitro radical, the other substituents of said compounds of formula (I) having the values indicated above,

ii) products of formula (I) belonging to formula (F):

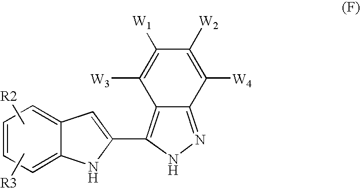

in which R2 and R3 represent the values indicated above and W3 and W4 both represent a hydrogen atom, then:

either W1 represents hydrogen and W2 does not represent aryl, heteroaryl or Y—X with Y chosen from O, S, C═CH2, C═O, S═O, SO2, alkylidene, NH and N(C1-C8)alkyl and X chosen from aryl, heteroaryl, NH(alkyl), NHcycloalkyl, NH(heterocycloalkyl), NH(aryl), NH(heteroaryl), NH(alkoxy) and NH(dialkylamide),

or W2 represents hydrogen and W1 does not represent alkyl, alkenyl, aryl, heteroaryl, carbocycle or heterocycle,

iii) products of formula (I) belonging to formula (FF):

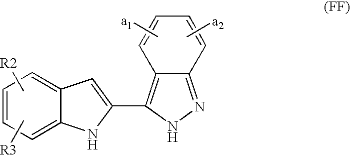

in which

R2 and R3, which may be identical or different, are chosen from the following values: hydrogen, COOalkyl, COOaryl, COOalkenyl, COOalkynyl, CO2H, halogen, OH, O-perfluoroalkyl, CONR7R8, CN, COOcycloalkyl, COOheterocyclyl, SO2NR7R8, SO2alkyl, which are optionally substituted,

it being understood that one from among R2 and R3 does not represent hydrogen,

and a1 and a2 are chosen from hydrogen, COOalkyl, COOaryl, COOalkenyl, COalkynyl, CO2H, halogen, OH, O-perfluoroalkyl, CONR7R8, CN, COOcycloalkyl, COOheterocyclyl, SO2NR7R8 and SO2alkyl, which are optionally substituted.

A subject of the present invention is thus the products of formula (I) as defined above in which R1 represents a pyrazolyl or indazolyl radical, these pyrazolyl or indazolyl radicals being optionally substituted with one or more radicals chosen from halogen atoms and hydroxyl, nitro, cyano, R4, OR4, SR4, —COR4, —OC(═O)R4, —C(═O)OR4, —C(═O)OH which is free or salified, —N(R5)C(═O)R4, —N(R5)C(═O)OR4, —S(O)nR4, —S(O)nOR4, —N(R5)SO2R4, —OS(O)nR4, —NY1Y2, —C(═O)NY1Y2, —OC(═O)NY1Y2, —N(R5)C(═O)NY1Y2, —S(O)nNY1Y2 and thienyl radicals, which are optionally substituted,

R2 and R3 are such that:

either R2 and R3, which may be identical or different, are chosen from a hydrogen atom, halogen atoms and hydroxyl, nitro, cyano, R4, —OR4, —COR4, —OC(═O)R4, —C(═O)OR4, —C(═O)OH, —N(R5)C(═O)R4, —N(R5)C(═O)OR4, —S(O)nR4, —S(O)nOR4, —N(R5)SO2R4, —NY1Y2, —C(═O)NY1Y2, —N(R5)C(═O)NY1Y2, —S(O)nNY1Y2 and —OC(═O)NY1Y2 radicals,

or R2 and R3 form, with the phenyl residue of the indole radical, a 4- to 6-membered carbon-based ring optionally containing one or more identical or different hetero atoms chosen from O, N and S, this ring being optionally substituted,

R4 represents alkyl, alk-NY1Y2, alk-CO—NY1Y2, alkenyl, alkynyl, cycloalkyl, aryl, heteroaryl, cycloalkylalkyl, heterocycloalkyl, heteroarylalkyl and arylalkyl, all these radicals being optionally substituted,

R5 represents hydrogen, alkyl, alkenyl, cycloalkyl, heterocycloalkyl, aryl, heteroaryl, arylalkyl, cycloalkylalkyl, heteroarylalkyl and heterocycloalkylalkyl, which are optionally substituted,

Y1 and Y2 are such that:

either Y1 and Y2, which may be identical or different, represent H, alkyl, alkenyl, cycloalkyl, heterocycloalkyl, heterocycloalkylalkyl, aryl, arylalkyl, arylcarboxy, heteroaryl, heteroarylalkyl and heteroarylcarboxy, all these radicals being optionally substituted,

or Y1 and Y2 form, together with the nitrogen atom to which they are attached, an optionally substituted cyclic amino radical,

all the alkyl, alkenyl, alkynyl, cycloalkyl, cycloalkylalkyl, heterocycloalkyl, heterocycloalkylalkyl, aryl, aryloxy, arylalkyl, arylcarboxy, heteroaryl, heteroarylalkyl and heteroarylcarboxy radicals being optionally substituted with one or more radicals chosen from halogen atoms and hydroxyl, alkoxy, alkyl, hydroxyalkyl, carboxyalkyl, cyano, nitro, trifluoromethyl, trifluoromethoxy, carboxy which is free, salified or esterified with an optionally substituted alkyl radical, —Nalk-COalk, —NH—COalk, S(O)n-alk, NH—S(O)n-alkyl, —NHCO—NY3Y4, —C(═O)—NY3Y4 and S(O)n-NY3Y4, aryl, arylalkoxy, aryloxy, aryloxyalkyl, heteroaryl and heterocycloalkyl radicals, which are optionally substituted,

with Y3 and Y4, which may be identical or different, representing hydrogen, alkyl or aryl, which are optionally substituted,

the latter alkyl (alk), heterocycloalkyl, aryl and heteroaryl radicals themselves being optionally substituted with one or more radicals chosen from halogen atoms and alkyl, carboxyl which is free, salified or esterified, amino, alkylamino, dialkylamino and phenylamino radicals,

all the phenyl radicals also being optionally substituted with a dioxole radical,

n represents an integer from 0 to 2,

alk represents alkyl of 1 to 6 carbon atoms,

said compounds of formula (I) being in all the possible racemic, enantiomeric and diastereoisomeric isomer forms, and also the addition salts with mineral and organic acids or with mineral bases.

A subject of the present invention is thus the products of formula (I) as defined above in which R1 represents a pyrazolyl or indazolyl radical, these radicals being optionally substituted with one or more radicals chosen from the values indicated in claim 1,

R2 and R3, which may be identical or different, are chosen from a hydrogen atom, halogen atoms and hydroxyl radicals, an alkyl radical optionally substituted with NY1Y2, alkenyl, —OR4, —CO—R4, —O—COR4, —OS(O)NR4, —O(CH2)n-CO—R4, nitro, cyano, aryl, heteroaryl and aryloxy radicals, a carboxyl radical, which free, salified or esterified with an alkyl radical optionally substituted or amidated with a radical NY1Y2 such that either Y1 and Y2, which may be identical or different, are chosen from H and alkyl, alkoxyalkyl, cycloalkyl, phenoxyalkyl, aryl, arylalkyl, cycloalkylalkyl, heterocycloalkylalkyl, heteroarylalkyl, arylcarboxyl and heteroarylcarboxyl radicals, which are optionally substituted, or Y1 and Y2 form, together with the nitrogen atom to which they are attached, an optionally substituted 5- or 6-membered cyclic radical, it being understood that R2 and R3 which are consecutive may form, with the indole radical to which they are attached, a 5- to 6-membered carbon-based ring containing one or more identical or different hetero atoms chosen from O, N and S,

R4 represents alkyl, alkenyl, cycloalkyl, aryl, heteroaryl and cycloalkylalkyl, which are optionally substituted,

all the alkyl, alkenyl, aryl, heteroaryl, aryloxy, cycloalkyl and heterocycloalkyl radicals contained in the above radicals being optionally substituted with one or more radicals chosen from halogen atoms and hydroxyl, alkoxy, alkyl, hydroxyalkyl, carboxyalkyl, cyano, nitro, trifluoromethyl, trifluoromethoxy, phenyl, thienyl, phenoxy, phenoxyalkyl, phenylalkoxy, —NH2, —NH(alk), —N(alk)2, —NH—SO2-alkyl, —NH(phenyl) and —NH(phenylalkyl) radicals, a carboxyl radical which is free, salified or esterified with an optionally substituted alkyl radical, —C(═O)—NH2, —C(═O)—NH(alk), C(═O)—N(alk)2, —NH—COalk, —C(═O)alk, —N(H)C(═O)alk, S(O)n-alk, S(O)n-NH2, S(O)n-NH(alk) and S(O)n-N(alk)2 radicals,

all the alkyl, alkenyl, alkoxy and alkylthio radicals above being linear or branched and containing not more than 6 carbon atoms,

all the phenyl radicals of the above radicals also being optionally substituted with a dioxole radical and one or more halogen atoms,

n represents an integer from 0 to 2,

it being understood that, when R1 represents an indazolyl radical to give the products of formula (I) below corresponding to formula (F):

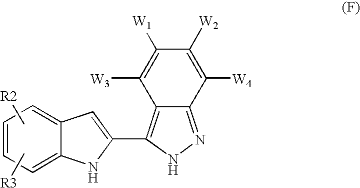

in which R2 and R3 represent the values indicated above and W3 and W4 both represent a hydrogen atom, then: either W1 represents hydrogen and W2 does not represent aryl, heteroaryl or Y—X with Y chosen from O, S, C═CH2, C═O, S═O, SO2, alkylidene, NH and N(C1-C8)alkyl and X chosen from aryl, heteroaryl, NH(alkyl), NHcycloalkyl, NH(heterocycloalkyl), NH(aryl), NH(heteroaryl), NH(alkoxy) and NH(dialkylamide),

or W2 represents hydrogen and W1 does not represent alkyl, alkenyl, aryl, heteroaryl, carbocycle or heterocycle,

said compounds of formula (I) being in all the possible racemic, enantiomeric and diastereoisomeric isomer forms, and also the addition salts with mineral and organic acids or with mineral and organic bases of said compounds of formula (I).

It is obvious that, depending on the ring represented by R1 and the number of ring members, R1 may comprise one to four substituents.

In the products of formula (I) and in the text hereinbelow:

the term “alkyl radical” denotes the linear and, where required, branched methyl, ethyl, propyl, isopropyl, butyl, isobutyl, sec-butyl, tert-butyl, pentyl, isopentyl, hexyl and isohexyl and also heptyl, octyl, nonyl and decyl radicals, and also the linear or branched positional isomers thereof,

the term “hydroxyalkyl radical” denotes the alkyl radicals indicated above substituted with at least one hydroxyl radical,

the term “alkenyl” denotes linear or branched radicals containing not more than 10 carbon atoms and one or more double bonds: mention may be made especially of vinyl, 1-propenyl, allyl, butenyl and 3-methyl-2-butenyl radicals, but also, for example, hepta-, octa-, nona- or deca-dienyl, for instance octa-2,6-dienyl, radicals,

the term “alkynyl” denotes linear or branched radicals containing not more than 10 carbon atoms: mention may be made especially of the alkyl radicals described above containing 2 to 10 carbon atoms and one or two triple bonds,

the term “alkylthio” denotes linear or branched radicals containing not more than 6 carbon atoms such as, especially, methylthio, ethylthio, propylthio, isopropylthio, butylthio, isobutylthio, sec-butylthio, tert-butylthio, pentylthio, isopentylthio, hexylthio or isohexylthio radicals, and also the linear or branched positional isomers thereof: among these alkylthio radicals, among those mentioned above, the ones preferably chosen are those containing not more than 4 carbon atoms. It is pointed out that all the alkylthio radicals are such that the sulphur atom is optionally oxidized to sulphone or sulphoxide with one or two oxygen atoms,

the term “alkoxy radical” denotes the linear and, where required, branched methoxy, ethoxy, propoxy, isopropoxy, linear, secondary or tertiary butoxy, pentoxy or hexoxy radicals containing not more than 10 carbon atoms, and also the linear or branched positional isomers thereof,

the term “alkenyloxy radical” denotes linear and branched —O-alkenyl radicals with alkenyl as defined above,

the terms “NH(alk)” and “N(alk)(alk)” denote amino radicals substituted, respectively, with one or two alkyl radicals, such alkyl radicals being linear or branched and chosen from alkyl radicals as defined above, preferably containing not more than 4 carbon atoms,

the term “acyl” denotes a radical R—C(O)— in which R represents a radical chosen from a hydrogen atom, linear or branched alkyl radicals containing not more than 6 carbon atoms, optionally substituted amino as defined above, and aryl, heteroaryl, cycloalkyl or heterocycloalkyl radicals, for example phenyl or pyrrolidinyl radicals: the term “acyl” thus especially denotes formyl radicals and acetyl, propionyl, butanoyl, pentanoyl, hexanoyl, benzoyl and pyrrolidinylcarbonyl radicals, for example,

the term “acylamino” denotes —C(O)—NH2, —C(O)—NH(alk) and —C(O)—N(alk)(alk) radicals: in these radicals, NH(alk) and N(alk)(alk) have the meanings given above,

the term “halogen atom” denotes chlorine, bromine, iodine or fluorine atoms and preferably the chlorine, bromine or fluorine atom,

the terms “aryl” and “heteroaryl” denote saturated radicals that are, respectively, carbocyclic and heterocyclic containing one or more hetero atoms, monocyclic or bicyclic that are not more than 12-membered,

the term “saturated or unsaturated carbocyclic or heterocyclic, monocyclic or bicyclic radical that is not more than 12-membered, containing one or more hetero atoms, which may be identical or different, chosen from O, N, NH and S, and which may contain a —C(O) member” includes the definitions which follow:

the term “unsaturated carbocyclic radical” especially denotes a cycloalkyl radical,

the term “cycloalkyl radical” denotes cyclopropyl, cyclobutyl, cyclopentyl and cyclohexyl radicals and most particularly cyclopentyl and cyclohexyl radicals,

the term “monocyclic heterocyclic radical” denotes a saturated or unsaturated 5- or 6-membered radical such that one or more of the members represents an oxygen, sulphur or nitrogen atom: such a heterocyclic or heterocycloalkyl radical thus denotes a carbocyclic radical interrupted with one or more hetero atoms chosen from oxygen, nitrogen and sulphur atoms, it being understood that the heterocyclic radicals can contain one or more hetero atoms chosen from oxygen, nitrogen and sulphur atoms and that, when these heterocyclic radicals contain more than one hetero atom, the hetero atoms of these heterocyclic radicals may be identical or different. Mention may be made especially of the dioxolane, dioxane, dithiolane, thiooxolane, thiooxane, morpholinyl, piperazinyl, piperazinyl substituted with a linear or branched alkyl radical containing not more than 4 carbon atoms, piperidyl, morpholinyl, thienyl such as 2-thienyl and 3-thienyl, furyl such as 2-furyl and 3-furyl, pyrimidinyl, pyridyl such as 2-pyridyl, 3-pyridyl and 4-pyridyl, pyrimidyl, pyrazolinyl, pyrrolyl, thiazolyl, isothiazolyl, diazolyl, thiadiazolyl, triazolyl, free or salified tetrazolyl, thiadiazolyl, thiatriazolyl, oxazolyl, oxadiazolyl or 3- or 4-isoxazolyl radicals. Mention may be made most particularly of morpholinyl, thienyl such as 2-thienyl and 3-thienyl, furyl such as 2-furyl, tetrahydrofuryl, thienyl, tetrahydrothienyl, hexahydropyran, pyrrolyl, pyrrolinyl, pyrazolinyl, isoxazolyl, pyridyl, pyrrolidinyl, imidazolyl, pyrazolyl, pyridazinyl and oxodihydropyridazinyl radicals,

the term “bicyclic heterocyclic radical” denotes a saturated (heteroaryl) or unsaturated 8- to 12-membered radical such that one or more of the members represents an oxygen, sulphur or nitrogen atom, and especially fused heterocyclic groups containing at least one hetero atom chosen from sulphur, nitrogen and oxygen, for example benzothienyl such as 3-benzothienyl, benzothiazolyl, quinolyl, isoquinolyl, tetralone, benzofuryl, dihydrobenzofuran, ethylenedioxyphenyl, thianthrenyl, benzopyrrolyl, benzimidazolyl, benzoxazolyl, thionaphthyl, indolyl, purinyl, indazolyl, thienopyrazolyl, tetrahydro-indazolyl, tetrahydrocyclopentapyrazolyl, dihydrofuro-pyrazolyl, tetrahydropyrrolopyrazolyl, oxotetrahydro-pyrrolopyrazolyl, tetrahydropyranopyrazolyl, tetrahydropyridinopyrazolyl or oxodihydropyridino-pyrazolyl,

the term “saturated carbocyclic radical” (aryl) especially denotes phenyl and naphthyl radicals and more particularly a phenyl radical. It may be noted that a carbocyclic radical containing a —C(O) member is, for example, a tetralone radical,

the term “alkylphenyl” denotes a phenyl radical substituted with one or more linear or branched alkyl radicals as defined above, preferably containing not more than 4 carbon atoms.

The carboxyl radical(s) in the products of formula (I) may be salified or esterified with various groups known to those skilled in the art, among which mention may be made, for example, of:

among the salification compounds, mineral bases such as, for example, one equivalent of sodium, potassium, lithium, calcium, magnesium or ammonium, or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine and N-methylglucamine,

among the esterification compounds, alkyl radicals to form alkoxycarbonyl groups such as, for example, methoxycarbonyl, ethoxycarbonyl, tert-butoxycarbonyl or benzyloxycarbonyl, these alkyl radicals possibly being substituted with radicals chosen, for example, from halogen atoms, hydroxyl, alkoxy, acyl, acyloxy, alkylthio, amino or aryl radicals, such as, for example, in chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) may be, for example, the salts formed with hydrochloric acid, hydrobromic acid, hydriodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulphonic acids such as, for example, methanesulphonic acid, ethanesulphonic acid or propanesulphonic acid, alkyldisulphonic acids such as, for example, methanedisulphonic acid and α,β-ethanedisulphonic acid, arylmonosulphonic acids such as benzenesulphonic acid, and aryldisulphonic acids.

It may be recalled that stereoisomerism may be defined in its broad sense as the isomerism of compounds having the same structural formulae, but whose various groups are arranged differently in space, such as especially in monosubstituted cyclohexanes in which the substituent may be in an axial or equatorial position, and the various possible rotational conformations of ethane derivatives. However, there is another type of stereoisomerism, due to the different spatial arrangements of attached substituents, either on double bonds or on rings, which is often referred to as geometrical isomerism or cis-trans isomerism. The term “stereoisomers” is used in the present patent application in its broadest sense and thus concerns all of the compounds indicated above.

A subject of the present invention is thus the products of formula (I) as defined above in which the substituents of said products of formula (I) are chosen from the values indicated above, and, especially, the aryl radicals represent phenyl and naphthyl radicals;

the heteroaryl radicals represent furyl, thienyl, benzothienyl, thianthrenyl, pyridyl, pyrazolyl, benzimidazolyl, benzofuran, isobenzofuran, dihydrobenzofuran, quinolyl, quinolone, adamantyl, isoxazolyl and dihydroquinolyl radicals; the cycloalkyl radicals represent cyclopropyl, cyclobutyl, cyclopentyl and cyclohexyl radicals; the heterocycloalkyl radicals represent hexahydropyran, piperidyl and morpholino radicals; the heterocycloalkylalkyl radicals represent hexahydropyranalkyl, piperidylalkyl and morpholinoalkyl radicals; the arylalkyl radicals represent phenylalkyl, ethylenedioxyphenylalkyl and naphthylalkyl radicals; the heteroarylalkyl radicals represent thienylalkyl, pyridylalkyl, furylalkyl, pyrazolylalkyl, benzothienylalkyl, dihydrobenzofuranalkyl and benzimidazolalkyl radicals; the aryloxy radicals represent phenoxy and naphthyloxy radicals; the arylalkoxy radicals represent phenylalkoxy and naphthylalkoxy radicals; the aryloxyalkyl radicals represent the phenoxyalkyl radical; all these radicals being optionally substituted as indicated above,

said compounds of formula (I) being in all the possible racemic, enantiomeric and diastereoisomeric isomer forms, and also the addition salts with mineral and organic acids or with mineral bases.

A subject of the present invention is thus the products of formula (I) as defined above in which R1 represents a pyrazolyl or indazolyl radical optionally substituted with one or two radicals chosen from halogen atoms and OH, R4, OR4, SR4, —COR4, —O—COR4, —OS(O)NR4, NO2, CN, CF3, OCF3, NY1Y2, free or salified or esterified carboxyl, —C(═O)—NY1Y2, —N(R5)C(═O)NY1Y2, —NH—CO—R4, S(O)n-alk, S(O)n-NY1Y2, —NR5-C(═O)R4, —NR5-S(O)nR4, —NR5-C(═O)OH, —NR5-C(═O)OR4, —OC(═O)NY1Y2 and thienyl radicals, all these radicals being optionally substituted,

R2 and R3, which may be identical or different, are chosen from a hydrogen atom; halogen atoms; hydroxyl radicals; alkyl optionally substituted with NY1Y2; alkenyl; alkoxy; nitro; cyano; furyl; thienyl; benzothienyl; naphthyl; thianthrenyl; phenyl; phenoxy and carboxyl which is free, salified or esterified with an alkyl radical or amidated with a radical NY1Y2, radicals, it being understood that R2 and R3 may form, with the indole radical to which they are attached, a 4,5-ethylenedioxybenzimidazole radical or a 4,5-methylenedioxybenzimidazole radical, which are optionally substituted,

with NY1Y2 such that either Y1 and Y2, which may be identical or different, are chosen from a hydrogen atom, alkyl; alkoxyalkyl; phenoxyalkyl; phenyl; phenylalkyl; phenylcarboxyl; naphthyl; naphthylalkyl; cycloalkylalkyl; cycloalkyl; furylalkyl; naphthylalkyl; thienylalkyl; piperidylalkyl; pyridylalkyl; benzothienylalkyl; pyrazolylalkyl; pyridylcarboxyl; dihydrobenzofuranalkyl; hexahydropyranalkyl; ethylenedioxyphenylalkyl; benzimidazolylalkyl radicals; all these radicals being optionally substituted,

or Y1 and Y2 form, together with the nitrogen atom to which they are attached, a pyrrolidinyl, pyrazolidinyl, pyrazolinyl, piperidyl, hexahydrofuran, morpholinyl or piperazinyl radical optionally substituted on the second nitrogen atom with an alkyl or phenyl radical, which are themselves optionally substituted,

R4 represents alkyl, alkenyl, cycloalkyl, phenyl and cycloalkylalkyl, which are optionally substituted,

R5 represents hydrogen, alkyl or phenyl, which is optionally substituted,

all the alkyl, alkenyl, phenyl, phenoxy, furyl, thienyl, piperidyl, pyridyl, pyrazolyl and benzimidazolyl radicals contained in the above radicals being optionally substituted with one or more radicals chosen from halogen atoms and hydroxyl, alkoxy, cyano, nitro, alkyl, hydroxyalkyl, carboxyalkyl, CF3, OCF3, NH2, NHalk, N(alk)2, NH(phenyl), NH(phenylalkyl), carboxyl which is free, salified or esterified with an alkyl radical, —C(═O)—NH2, —C(═O)—NH(alk), C(═O)—N(alk)2, NH—COalk, —C(═O)alk, S(O)n-alk, S(O)n-NH2, S(O)n-NH(alk), S(O)n-N(alk)2, thienyl, phenylalkyl, phenoxyalkyl, phenoxy, phenylalkoxy, morpholino, piperidyl and phenyl radicals, in all these radicals the phenyl radical itself being optionally substituted with one or more radicals chosen from halogen atoms and cyano, CF3, OCF3, alkyl, phenyl-S(O)n-alk-phenyl, alkoxy, NH2, NHalk, N(alk)2, SO2NH2, SO2Nalk and SO2N(alk)2 radicals,

with n representing an integer from 0 to 2,

all the alkyl, alkenyl, alkoxy and alkylthio radicals above being linear or branched and containing not more than 6 carbon atoms,

all the phenyl radicals of the above radicals also being optionally substituted with a dioxole radical, said compounds of formula (I) being in all the possible racemic, enantiomeric and diastereoisomeric isomer forms, and also the addition salts with mineral and organic acids or with mineral and organic bases of said compounds of formula (I).

One subject of the present invention is thus the compounds of formula (I) as defined above in which R1 represents a pyrazolyl or indazolyl radical optionally substituted with one or more radicals chosen from halogen atoms and R4, OR4, SR4, thienyl, —N(R5)C(═O)R4, —N(R5)SO2R4, —NY1Y2, —C(═O)NY1Y2 or —NH—C(═O)NY1Y2 radicals,

R2 and R3, which may be identical or different, are chosen from a hydrogen atom, halogen atoms, hydroxyl, alkyl and alkoxy, nitro, cyano, phenyl and phenoxy radicals, a carboxyl radical which is free, salified or esterified with an alkyl radical or amidated with a identical or different, are chosen from a hydrogen atom and alkyl, phenyl, phenylalkyl, cycloalkylalkyl, cycloalkyl, furylalkyl and pyridylcarboxyl radicals,

or Y1 and Y2 form, together with the nitrogen atom to which they are attached, a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,

R4 represents alkyl, cycloalkyl, phenyl and cycloalkylalkyl, which are optionally substituted,

R5 represents a hydrogen atom or an optionally substituted alkyl,

all the alkyl, alkoxy, phenyl and phenoxy radicals indicated above being optionally substituted with one or more radicals chosen from halogen atoms and hydroxyl, cyano, alkyl, alkoxy, free, salified or esterified carboxyl, NH2, NHAlk, N(Alk)2, NHSO2Alk, phenylamino, phenylalkylamino, phenyl, morpholino, furyl and pyridyl radicals,

all the alkyl, Alk and alkoxy radicals mentioned above being linear or branched and containing not more than 6 carbon atoms,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is thus particularly the products of formula (I) as defined above corresponding to formula (I) in which R1, R2 and R3 are among the meanings indicated above, with NY1Y2 such that either Y1 and Y2, which may be identical or different, are chosen from a hydrogen atom and alkyl, phenyl, phenylalkyl, cycloalkylalkyl, cycloalkyl, furylalkyl and pyridylcarboxyl radicals,

or Y1 and Y2 form, together with the nitrogen atom to which they are attached, a pyrrolidinyl, morpholino or piperazinyl radical optionally substituted on the second nitrogen atom with an alkyl or phenyl radical, which are themselves optionally substituted with an NH2, NHAlk, N(Alk)2 or NHSO2Alk radical, or a morpholino, furyl or pyridyl radical,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is thus the products of formula (I) as defined above in which R1 represents a pyrazolyl radical optionally substituted with one or two substituents chosen from the values indicated above, the other substituents R2, R3, R4 and R5 being chosen from the values defined above,

said compounds of formula (I) being in all the possible racemic, enantiomeric and diastereoisomeric isomer forms, and also the addition salts with mineral and organic acids or with mineral and organic bases of said compounds of formula (I).

Among the compounds of formula (I) above that will particularly be noted are those for which R1 represents a pyrazolyl radical that is unsubstituted on its NH nitrogen atom and optionally substituted with one or two substituents chosen from the values indicated above, the other substituents R2, R3, R4 and R5 being chosen from the values defined above,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is especially the products of formula (I) as defined above in which R1 represents a pyrazolyl radical corresponding to formula (P):

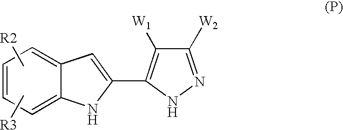

in which R2 and R3 represent the values indicated in any one of the preceding claims and W1 and W2 are such that:

either W1 and W2, which may be identical or different, are chosen from hydrogen, OR4, SR4, —N(R5)C(═O)R4, —N(R5)SO2R4, —NY1Y2, —N(R5)C(═O)NY1Y2 and —C(═O)NY1Y2, or one of W1 and W2 represents hydrogen, OR4 or SR4 and the other among W1 and W2 represents hydrogen, —N(R5)C(═O)R4, —N(R5)SO2R4, —NY1Y2, —N(R5)C(═O)NY1Y2 or —C(═O)NY1Y2,

or W1 represents hydrogen, —N(R5)C(═O)R4, —N(R5)SO2R4, —NY1Y2(NH2), —N(R5)C(═O)NY1Y2 or —C(═O)NY1Y2 and W2 represents hydrogen, OR4 or SR4,

it being understood that W1 and W2 do not both represent hydrogen,

with R4, R5, Y1 and Y2 as defined above,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is especially the compounds of formula (I) as defined above in which R1 represents a pyrazolyl radical substituted with two substituents W1 and W2 such that one represents hydrogen, OR4 or SR4 and the other represents hydrogen, —N(R5)C(═O)R4, —N(R5)SO2R4, —NY1Y2, —C(═O)NY1Y2 or —NH—C(═O)NY1Y2,

it being understood that W1 and W2 do not both represent hydrogen,

with R4 representing alkyl, cycloalkyl or phenyl, which are optionally substituted,

R5 represents an optionally substituted hydrogen atom or an optionally substituted alkyl,

NY1Y2 being such that either Y1 and Y2, which may be identical or different, are chosen from a hydrogen atom and optionally substituted alkyl and pyridylcarboxyl radicals, or Y1 and Y2 form, together with the nitrogen atom to which they are attached, a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,

all the alkyl, alkoxy and phenyl radicals indicated above also being optionally substituted with an NH2, NHAlk, N(Alk)2 or NHSO2Alk radical or a morpholino, furyl or pyridyl radical or a phenyl radical itself optionally substituted with one or more radicals chosen from halogen atoms and alkyl, salified or esterified free carboxyl, amino, alkylamino, dialkylamino, phenylamino, hydroxyl, alkoxy and NHCOalk radicals,

all the alkyl, Alk and alkoxy radicals indicated above being linear or branched and containing not more than 6 carbon atoms,

all the pyridyl radicals themselves being optionally substituted with a halogen atom,

R2 and R3 being chosen from the values defined in any one of the preceding claims,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is, more particularly, the compounds of formula (I) as defined above in which R1 represents a pyrazolyl radical substituted with two substituents W1 and W2 such that one represents hydrogen, OR4 or SR4 and the other represents hydrogen, —N(R5)C(═O)R4, —N(R5)SO2R4, —NY1Y2, —C(═O)NY1Y2 or —NH—C(═O)NY1Y2,

it being understood that W1 and W2 do not both represent hydrogen,

with R4 representing alkyl, cycloalkyl or phenyl, which are optionally substituted,

R5 represents an optionally substituted hydrogen atom or an optionally substituted alkyl,

NY1Y2 being such that either Y1 and Y2, which may be identical or different, are chosen from a hydrogen atom and optionally substituted alkyl and pyridylcarboxyl radicals, or Y1 and Y2 form, together with the nitrogen atom to which they are attached, a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,

all the alkyl, alkoxy and phenyl radicals indicated above also being optionally substituted with an NH2, NHAlk, N(Alk)2 or NHSO2Alk radical or a morpholino, furyl or pyridyl radical,

all the alkyl, Alk and alkoxy radicals indicated above being linear or branched and containing not more than 6 carbon atoms,

all the pyridyl radicals themselves being optionally substituted with a halogen atom,

R2 and R3 being chosen from the values defined in any one of the claims,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is, most particularly, the compounds of formula (I) as defined above in which R1 represents a pyrazolyl radical substituted with two substituents W1 and W2 as defined above, such that one represents a hydrogen atom and the other represents a radical OR4

with R4 representing alkyl, cycloalkyl or phenyl radicals optionally substituted with an NH2, NHAlk, N(Alk)2 or NHSO2Alk radical, a morpholino, furyl, pyridyl radical or a phenyl radical itself optionally substituted with one or more radicals chosen from halogen atoms and amino, alkylamino, dialkylamino, phenylamino, hydroxyl, alkoxy and NHCOalk radicals,

all the alkyl, Alk and alkoxy radicals indicated above being linear or branched and containing not more than 6 carbon atoms,

R2 and R3 being chosen from the values defined above, said compounds of formula (I) being in all the possible racemic, enantiomeric and diastereoisomeric isomer forms, and also the addition salts with mineral and organic acids or with mineral and organic bases of said compounds of formula (I).

A subject of the present invention is thus the products of formula (I) as defined above in which R1 represents an indazolyl radical optionally substituted with one or more substituents chosen from the values indicated above,

the other substituents R2, R3, R4 and R5 being chosen from the values defined in any one of the claims,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is thus, particularly, the products of formula (I) as defined above in which R1 represents an indazolyl radical,

R2 and R3 are such that one represents a hydrogen atom and the other is chosen from the following radicals: a hydrogen atom, halogen atoms, alkyl radicals optionally substituted with a radical NY1Y2, alkoxy, cyano and carboxyl which is free, salified or esterified with an alkyl radical or amidated as a radical CONY1Y2,

NY1Y2 being such that either Y1 and Y2, which may be identical or different, are chosen from a hydrogen atom and alkyl and pyridylcarboxyl radicals, or Y1 and Y2 form, with the nitrogen atom to which they are attached, a pyrrolidinyl, pyrazolidinyl, pyrazolinyl, piperidyl or morpholino radical or a piperazinyl radical optionally substituted with an alkyl or phenyl radical, which are themselves optionally substituted,

all the alkyl, alkoxy and phenyl radicals indicated above also being optionally substituted with an NH2, NHAlk, N(Alk)2 or NHSO2Alk radical or a morpholino, furyl or pyridyl radical,

Alk meaning alkyl,

all the alkyl, Alk and alkoxy radicals indicated above being linear or branched and containing not more than 4 carbon atoms,

all the pyridyl radicals themselves being optionally substituted with a halogen atom,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is thus, most particularly, the compounds of formula (I) as defined above in which R1 represents an indazolyl radical,

R2 and R3 are such that one represents a hydrogen atom and the other is chosen from the following radicals: a hydrogen atom, halogen atoms, alkyl radicals optionally substituted with a radical NY1NY2, alkoxy radicals optionally substituted with a morpholino radical, a cyano radical and a carboxyl radical which is free, salified or esterified with an alkyl radical or amidated as a radical CONY1Y2,

NY1Y2 being such that either Y1 and Y2, which may be identical or different, are chosen from a hydrogen atom and alkyl, furylalkyl, pyridylcarboxyl and pyridylalkyl radicals in which the pyridyl radicals are themselves optionally substituted with a halogen atom, or Y1 and Y2 form, with the nitrogen atom to which they are attached, a piperazinyl radical optionally substituted with an alkyl or phenyl radical, which are themselves optionally substituted with an NHSO2CH3, NH2, NHAlk or N(Alk)2 radical,

all the alkyl or Alk and alkoxy radicals indicated above being linear or branched and containing not more than 4 carbon atoms,

said compounds of formula (I) being in all the possible racemic, enantiomeric and diastereoisomeric isomer forms, and also the addition salts with mineral and organic acids or with mineral and organic bases of said compounds of formula (I).

A subject of the present invention is particularly the products of formula (I) as defined above, corresponding to the following formulae:

3-(5-cyanoindol-2-yl)indazole

3-(indol-2-yl)indazole

3-(5-ethoxycarbonylindol-2-yl)indazole

3-(5-(N,N-diisopropyl)carboxamideindol-2-yl)indazole

3-(5-methylindol-2-yl)indazole

3-(5-chloroindol-2-yl)indazole

3-(6-methylindol-2-yl)indazole

3-(5-carboxyindol-2-yl)indazole

3-(5-(N-(2-chloropyridin-5-yl)methyl)carboxamideindol-2-yl)indazole

3-(5-(morpholinoethyloxy)indol-2-yl)indazole

3-(5-aminomethylindol-2-yl)indazole

3-(5-(N-((2-furyl)methyl))carboxamideindol-2-yl)indazole

3-(6-methoxycarbonylindol-2-yl)indazole

3-(5-((2-chloropyrid-5-yl)carboxamido)methylene)indol-2-yl)indazole

3-(6-carboxyindol-2-yl)indazole

3-(6-(N-(2-chloropyrid-5-yl)methyl)carboxamideindol-2-yl)indazole

3-(6-(N-((2-furyl)methyl))carboxamideindol-2-yl)indazole

3-(5-(N-(4-methylsulphonamidephenyl)piperazinocarboxamide)indol-2-yl)indazole

4-amino-3-(indol-2-yl)pyrazole.

A subject of the present invention is, most particularly, the products of formula (I) as defined above, corresponding to the following formulae:

3-(5-ethoxycarbonylindol-2-yl)indazole

3-(5-(N,N-diisopropyl)carboxamideindol-2-yl)indazole

3-(5-methylindol-2-yl)indazole

3-(5-chloroindol-2-yl)indazole

3-(6-methylindol-2-yl)indazole

3-(5-carboxyindol-2-yl)indazole

3-(5-(N-(2-chloropyrid-5-yl)methyl)carboxamideindol-2-yl)indazole

3-(5-(morpholinoethyloxy)indol-2-yl)indazole

3-(5-aminomethylindol-2-yl)indazole

3-(5-(N-((2-furyl)methyl))carboxamideindol-2-yl)indazole

3-(5-((2-chloropyrid-5-yl)carboxamido)methylene)indol-2-yl)indazole

3-(6-(N-(2-chloropyrid-5-yl)methyl)carboxamideindol-2-yl)indazole

3-(6-(N-((2-furyl)methyl))carboxamideindol-2-yl)indazole

3-(5-(N-(4-methylsulphonamidephenyl)-piperazinocarboxamide)indol-2-yl)indazole

4-amino-3-(indol-2-yl)pyrazole

3-[5-(1H-indol-2-yl)-2H-pyrazol-3-yloxymethyl]phenol

N-{3-[5-(indol-2-yl)-2H-pyrazol-3-yloxymethyl]phenyl}acetamide

2-[5-(3-fluorobenzyloxy)-1H-pyrazol-3-yl]-1H-indole.

A subject of the present invention is also processes for preparing the products of formula (I) as defined above.

The scheme (1) below in particular gives a general process for synthesizing the products of formula (I) as defined above for which R1 thus represents a pyrazolyl or indazolyl radical.

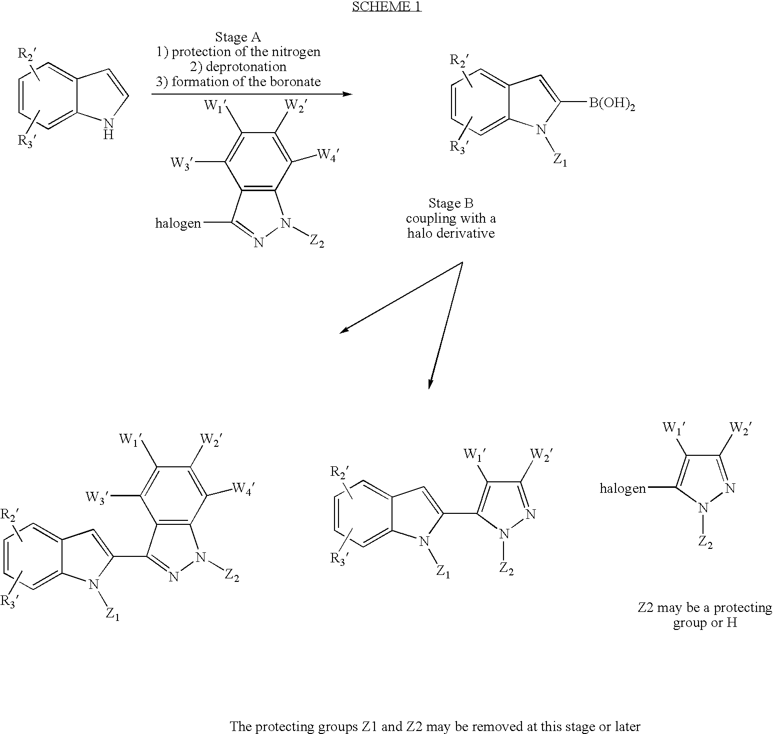

In this scheme (1):

R2′ and R3′ represent the values of R2 and R3 as defined above for the products of formula (I), in which the possible reactive functions are optionally protected.

W1′, W2′, W3′ and W4′ represent the values of W1, W2, W3 and W4 as defined above in which the possible reactive functions are optionally protected, W1, W2, W3 and W4 thus representing the possible substituents of R1 as defined above for the products of formula (I), i.e. W1 and W2 when R1 represents a pyrazolyl radical and W1, W2, W3 and W4 when R1 represents an indazolyl radical.

The scheme (2) below also gives a general process for synthesizing the products of formula (I) as defined above for which R1 thus represents a pyrazolyl radical.

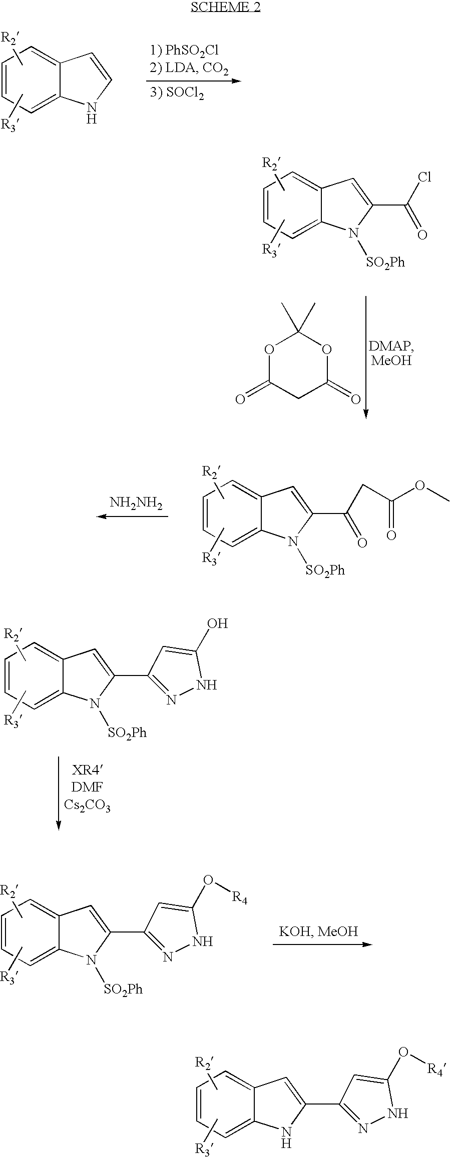

In this scheme (2):

R2′ and R3′ represent the values of R2 and R3 as defined above for the products of formula (I), in which the possible reactive functions are optionally protected.

R4′ represents the values of R4 as defined above for the products of formula (I), in which the possible reactive functions are optionally protected.

An illustration of the above process is given in the preparation of Examples 20 to 22 of the present invention.

Such products obtained by the above schemes may be products of formula (I) or alternatively intermediates to obtain products of formula (I) or products to be converted into other products of formula (I), may be subjected, if desired and if necessary, to one or more of the following conversion reactions, in any order:

a) an esterification or amidation reaction of an acid function,

b) a saponification reaction of an ester function to an acid function,

c) an oxidation reaction of an alkylthio group to the corresponding sulphoxide or sulphone,

d) a reaction for conversion of a ketone function to an oxime function,

e) a reaction for reduction of the free or esterified carboxyl function to an alcohol function,

f) a reaction for conversion of the alkoxy function to a hydroxyl function, or alternatively of the hydroxyl function to an alkoxy function,

g) a reaction for oxidation of an alcohol function to an aldehyde, acid or ketone function,

h) a reaction for conversion of a nitrile radical to tetrazolyl,

i) a reaction for removal of the protecting groups that may be borne on the protected reactive functions,

j) a salification reaction with a mineral or organic acid or with a base to give the corresponding salt,

k) a reaction for resolution of the racemic forms into resolved products,

said compounds of formula (I) thus obtained being in any possible racemic, enantiomeric or diastereoisomeric isomer form.

It may be noted that such conversion reactions of substituents into other substituents may also be carried out on the starting materials, and also on the intermediates as defined above before continuing the synthesis according to the reactions indicated in the process described above.

The process described above may be performed under the usual conditions known to those skilled in the art and especially under the reaction conditions described below for the preparation of the examples of the present patent application.

Especially, in stage A of the above scheme, the process is preferably performed in the following manner:

Nitrogen protection step: using protecting groups known to those skilled in the art, such as the Boc group, working, for example, in the presence of a mineral base (for example NaHCO₃) or an organic base (for example DMAP) in an inert organic solvent at a temperature in the region of 20° C.

Step of deprotection and formation of the boronate: using a base such as LDA (lithium diisopropylamide, at a temperature in the region of 0° C., in an inert organic solvent such as tetrahydrofuran, and using [lacuna].

In stage B of the above scheme especially, the process is preferably performed in the presence of a mineral base such as sodium bicarbonate, in the presence of a catalyst such as palladium complexed with triphenylphosphine, in an inert organic solvent such as toluene or DMF, at a temperature of between room temperature and the reflux point of the reaction medium.

Among the starting materials used for the preparation of the products of formula (I) according to the present invention, some are known and may be obtained commercially or may be prepared according to the usual methods known to those skilled in the art.

Certain starting materials may also especially be prepared from commercial products, for example by subjecting them to one or more of the reactions described above in a) to k), performed under the conditions that are also described above.

The experimental section below gives examples of such starting materials.

The following references are also cited, which may be used for the preparation of benzimidazoles, pyrazoles or indazoles in the context of the present invention:

G. R. Newkome, W. W. Paudler, Contemporary Heterocyclic Chemistry, Syntheses, Reactions and Applications, J. Wiley, 1982

Behr, Fusco, Jarboe, Heterocyclic Compounds, Pyrazoles, Pyrazolines, Pyrazolidines, indazoles and condensed rings, J. Wiley, 1967.

For the preparation of the products of formula (I) according to the present invention, the various reactive functions which may be borne by some of the compounds in the reactions defined above may, if necessary, be protected: these are, for example, hydroxyl, acyl, free carboxyl or amino and monoalkylamino radicals, which may be protected with suitable protecting groups.

The following non-exhaustive list of examples of protection of reactive functions may be cited:

the hydroxyl groups may be protected, for example, with alkyl radicals such as tert-butyl, trimethylsilyl, tert-butyldimethylsilyl, methoxymethyl, tetrahydropyranyl, benzyl or acetyl,

the amino groups may be protected, for example, with acetyl, trityl, benzyl, tert-butoxycarbonyl, BOC, benzyloxycarbonyl or phthalimido radicals or other radicals known in peptide chemistry,

the acyl groups such as the formyl group may be protected, for example, in the form of cyclic or acyclic ketals or thioketals, such as dimethyl or diethyl ketal or ethylenedioxy ketal, or diethylthio ketal or ethylenedithio ketal,

the acid functions in the products described above may, if desired, be amidated with a primary or secondary amine, for example in methylene chloride in the presence, for example, of 1-ethyl-3-(dimethylaminopropyl)carbodiimide hydrochloride at room temperature;

the acid functions may be protected, for example, in the form of esters formed with readily cleavable esters such as benzyl or tert-butyl esters or esters known in peptide chemistry.

The reactions a) to k) may be performed, for example, as indicated below.

a) The products described above may, if desired, be subjected, on the possible carboxyl functions, to esterification or amidation reactions which may be performed according to the usual methods known to those skilled in the art. The amidation reactions may especially be performed in the presence of a coupling agent such as a carbodiimide derivative. Examples that may be mentioned include N-(3-dimethylaminopropyl)-N′-ethylcarbodiimide (EDCI), N,N′-diisopropylcarbodiimide (DIC) and N,N′-dicyclohexylcarbodiimide.

b) The possible conversions of ester functions into acid functions in the products described above may, if desired, be performed under the usual conditions known to those skilled in the art, especially by acid or alkaline hydrolysis, for example with sodium hydroxide or potassium hydroxide in alcoholic medium such as, for example, in methanol, or alternatively with hydrochloric acid or sulphuric acid.

The saponification reaction may be performed according to the usual methods known to those skilled in the art, such as, for example, in a solvent such as methanol or ethanol, dioxane or dimethoxyethane, in the presence of sodium hydroxide or potassium hydroxide.

c) The possible alkylthio groups in the products described above may, if desired, be converted into the corresponding sulphoxide or sulphone functions under the usual conditions known to those skilled in the art, such as, for example, with peracids such as, for example, peracetic acid or meta-chloroperbenzoic acid or alternatively with ozone, oxone or sodium periodate in a solvent such as, for example, methylene chloride or dioxane at room temperature.

The production of the sulphoxide function may be promoted by an equimolar mixture of the product containing an alkylthio group and of a reagent such as, especially, a peracid.

The production of the sulphone function may be promoted by a mixture of the product containing an alkylthio group with an excess of a reagent such as, especially, a peracid.

d) The reaction for the conversion of a ketone function to an oxime may be performed under the usual conditions known to those skilled in the art, such as, especially, an action in the presence of an optionally O-substituted hydroxylamine in an alcohol such as, for example, ethanol, at room temperature or with heating.

e) The possible free or esterified carboxyl functions in the products described above may, if desired, be reduced to an alcohol function by the methods known to those skilled in the art: the possible esterified carboxyl functions may, if desired, be reduced to an alcohol function by the methods known to those skilled in the art and especially with lithium aluminium hydride in a solvent such as, for example, tetrahydrofuran or dioxane or ethyl ether.

The possible free carboxyl functions in the products described above may, if desired, be reduced to an alcohol function especially with boron hydride.

f) The possible alkoxy functions such as, especially, methoxy in the products described above may, if desired, be converted into a hydroxyl function under the usual conditions known to those skilled in the art, for example with boron tribromide in a solvent such as, for example, methylene chloride, with pyridine hydrobromide or hydrochloride or alternatively with hydrobromic acid or hydrochloric acid in water or trifluoroacetic acid at reflux.

g) The possible alcohol functions in the products described above may, if desired, be converted into an aldehyde or acid function by oxidation under the usual conditions known to those skilled in the art, such as, for example, by the action of manganese oxide to give aldehydes, or Jones reagent to give acids.

h) The possible nitrile functions in the products described above, may, if desired, be converted into tetrazolyl under the usual conditions known to those skilled in the art, such as, for example, by cycloaddition of a metal azide such as, for example, sodium azide or a trialkyltin azide with the nitrile function, as indicated in the method described in the article referenced as follows:

J. Organometallic Chemistry, 33, 337 (1971) KOZIMA S. et al.

It may be noted that the reaction for the conversion of a carbamate to a urea and especially of a sulphonylcarbamate to a sulphonylurea may be performed, for example, in a refluxing solvent such as, for example, toluene in the presence of a suitable amine.

It is understood that the reactions described above may be carried out as indicated or alternatively, where appropriate, according to other usual methods known to those skilled in the art.

i) The removal of protecting groups such as, for example, those indicated above may be carried out under the usual conditions known to those skilled in the art, especially by an acid hydrolysis performed with an acid such as hydrochloric acid, benzenesulphonic acid or paratoluenesulphonic acid, formic acid or trifluoroacetic acid, or alternatively by a catalytic hydrogenation.

The phthalimido group may be removed with hydrazine.

A list of the various protecting groups that may be used will be found, for example, in patent BF 2 499 995.

j) The products described above may, if desired, be subjected to salification reactions, for example with a mineral or organic acid or with a mineral or organic base according to the usual methods known to those skilled in the art: such a salification reaction may be performed, for example, in the presence of hydrochloric acid, for example, or alternatively tartaric acid, citric acid or methanesulphonic acid, in an alcohol such as, for example, ethanol or methanol.

k) The possible optically active forms of the products described above may be prepared by resolution of the racemic mixtures according to the usual methods known to those skilled in the art.

Illustrations of such reactions defined above are given in the preparation of the examples described below. The products of formula (I) as defined above and also the addition salts thereof with acids have advantageous pharmacological properties, especially on account of their kinase-inhibiting properties as indicated above.

It may be indicated that since certain protein kinases have a central role in the initiation, development and completion of events of the cell cycle, molecules that inhibit such kinases are capable of limiting unwanted cell proliferations such as those observed in cancers, and can intervene in the prevention, regulation or treatment of neurodegenerative diseases such as Alzheimer's disease or neuronal apoptosis.

The products of the present invention are most particularly useful for preventing, regulating or treating diseases requiring anti-angiogenic activity.

The products of the present invention are especially useful for tumour therapy.

The products of the invention can thus also increase the therapeutic effects of commonly-used anti-tumoral agents.

The products of formula (I) of the present invention thus most particularly have anti-angiogenic properties.

These properties justify their therapeutic use, and the subject of the invention is, particularly, as medicinal products, the compounds of formula (I) as defined above, said compounds of formula (I) being in any possible racemic, enantiomeric or diastereoisomeric isomer form, and also the addition salts with pharmaceutically acceptable mineral and organic acids or with pharmaceutically acceptable mineral and organic bases of said compounds of formula (I).

One subject of the invention is thus, more particularly, as medicinal products, the products as defined by formula (I), said compounds of formula (I) being in any possible racemic, enantiomeric or diastereoisomeric isomer form, and also the addition salts with pharmaceutically acceptable mineral and organic acids or with pharmaceutically acceptable mineral and organic bases of said compounds of formula (I).

One subject of the invention is particularly, as medicinal products, the products described below in the examples and especially the products corresponding to the following formulae:

3-(5-cyanoindol-2-yl)indazole

3-(indol-2-yl)indazole

3-(5-ethoxycarbonylindol-2-yl)indazole

3-(5-(N,N-diisopropyl)carboxamideindol-2-yl)indazole

3-(5-methylindol-2-yl)indazole

3-(5-chloroindol-2-yl)indazole

3-(6-methylindol-2-yl)indazole

3-(5-carboxyindol-2-yl)indazole

3-(5-(N-(2-chloropyrid-5-yl)methyl)carboxamideindol-2-yl)indazole

3-(5-(morpholinoethyloxy)indol-2-yl)indazole

3-(5-aminomethylindol-2-yl)indazole

3-(5-(N-((2-furyl)methyl))carboxamideindol-2-yl)indazole

3-(6-methoxycarbonylindol-2-yl)indazole

3-(5-((2-chloropyrid-5-yl)carboxamido)methylene)indol-2-yl)indazole

3-(6-carboxyindol-2-yl)indazole

3-(6-(N-(2-chloropyrid-5-yl)methyl)carboxamideindol-2-yl)indazole

3-(6-(N-((2-furyl)methyl))carboxamideindol-2-yl)indazole

3-(5-(N-(4-methylsulphonamidephenyl)-piperazinocarboxamide)indol-2-yl)indazole

4-amino-3-(indol-2-yl)pyrazole

3-[5-(1H-indol-2-yl)-2H-pyrazol-3-yloxymethyl]phenol

N-{3-[5-(indol-2-yl)-2H-pyrazol-3-yloxymethyl]phenyl}acetamide

2-[5-(3-fluorobenzyloxy)-1H-pyrazol-3-yl]-1H-indole.

A subject of the present invention is, most particularly, as medicinal products, the products of formula (I) as defined above, corresponding to the following formulae:

3-(5-ethoxycarbonylindol-2-yl)indazole

3-(5-(N,N-diisopropyl)carboxamideindol-2-yl)indazole

3-(5-methylindol-2-yl)indazole

3-(5-chloroindol-2-yl)indazole

3-(6-methylindol-2-yl)indazole

3-(5-carboxyindol-2-yl)indazole

3-(5-(N-(2-chloropyrid-5-yl)methyl)carboxamideindol-2-yl)indazole

3-(5-(morpholinoethyloxy)indol-2-yl)indazole

3-(5-aminomethylindol-2-yl)indazole

3-(5-(N-((2-furyl)methyl))carboxamideindol-2-yl)indazole

3-(5-((2-chloropyrid-5-yl)carboxamido)methylene)indol-2-yl)indazole

3-(6-(N-(2-chloropyrid-5-yl)methyl)carboxamideindol-2-yl)indazole

3-(6-(N-((2-furyl)methyl))carboxamideindol-2-yl)indazole

3-(5-(N-(4-methylsulphonamidephenyl)piperazino-carboxamide)indol-2-yl)indazole

4-amino-3-(indol-2-yl)pyrazole

3-[5-(1H-indol-2-yl)-2H-pyrazol-3-yloxymethyl]phenol

N-{3-[5-(indol-2-yl)-2H-pyrazol-3-yloxymethyl]phenyl}acetamide

2-[5-(3-fluorobenzyloxy)-1H-pyrazol-3-yl]-1H-indole.

The invention also relates to pharmaceutical compositions containing, as active principle, at least one of the products of formula (I) as defined above, or a pharmaceutically acceptable salt of this product or a prodrug of this product and, where appropriate, a pharmaceutically acceptable support.

The invention thus covers pharmaceutical compositions containing, as active principle, at least one of the medicinal products as defined above.

Such pharmaceutical compositions of the present invention can also, where appropriate, contain active principles of other antimitotic medicinal products such as, in particular, those based on taxol, cis-platin, DNA-intercalating agents and the like.

These pharmaceutical compositions may be administered orally, parenterally or locally by topical application to the skin and mucous membranes or by intravenous or intramuscular injection.

These compositions may be solid or liquid and may be in any pharmaceutical form commonly used in human medicine, such as, for example, simple or sugar-coated tablets, pills, lozenges, gel capsules, drops, granules, injectable preparations, ointments, creams or gels; they are prepared according to the usual methods. The active principle may be incorporated therein with excipients usually used in these pharmaceutical compositions, such as talc, gum arabic, lactose, starch, magnesium stearate, cocoa butter, aqueous or non-aqueous vehicles, fatty substances of animal or plant origin, paraffin derivatives, glycols, and various wetting agents, dispersants, emulsifiers or preserving agents.

The usual dosage, which is variable depending on the product used, the individual treated and the complaint under consideration, may be, for example, from 0.05 to 5 g per day in adults, or preferably from 0.1 to 2 g per day.

The subject of the present invention is also the use of the products of formula (I) as defined above, or of pharmaceutically acceptable salts of these products, for the preparation of a medicinal product intended for inhibiting the activity of a protein kinase.

A subject of the present invention is also the use of products of formula (I) as defined above for the preparation of a medicinal product for treating or preventing a disease characterized by deregulation of the activity of a protein kinase.

Such a medicinal product may especially be intended for treating or preventing a disease in a mammal.

A subject of the present invention is also the use defined above, in which the protein kinase is a tyrosine kinase protein.

A subject of the present invention is also the use defined above, in which the protein kinase is chosen from the following group: EGFR, Fak, FLK-1, FGFR1, FGFR2, FGFR3, FGFR4, FGFR5, flt-1, IGF-1R, KDR, PLK, PDGFR, tie2, VEGFR, AKT, Raf and Aurora 1 or 2.

A subject of the present invention is, more particularly, the use defined above in which the protein kinase is chosen from KDR, Fak, tie2, Aurora, AKT and IGF-1R.

A subject of the present invention is especially the use defined above, in which the kinase protein is KDR. A subject of the present invention is also the use defined above, in which the protein kinase is in a cell culture.

A subject of the present invention is also the use defined above, in which the protein kinase is in a mammal.

A subject of the present invention is particularly the use of a product of formula (I) as defined above, for the preparation of a medicinal product for treating or preventing a disease chosen from the following group: disorders of the proliferation of blood vessels, fibrotic disorders, disorders of the proliferation of “mesangial” cells, metabolic disorders, allergies, asthma, thrombosis, diseases of the nervous system, retinopathy, psoriasis, rheumatoid arthritis, diabetes, muscle degeneration and cancers.

A subject of the present invention is, more particularly, the use of a product of formula (I) as defined above, for the preparation of a medicinal product for treating or preventing a disease chosen from the following group: disorders of the proliferation of blood vessels, fibrotic disorders, disorders of the proliferation of “mesangial” cells, retinopathy, psoriasis, rheumatoid arthritis, diabetes, muscle degeneration and cancers.

A subject of the present invention is, most particularly, the use of a product of formula (I) as defined above, for the preparation of a medicinal product for preventing or treating diseases associated with an uncontrolled angiogenesis, for the preparation of a medicinal product for treating oncology diseases and especially intended for the treatment of cancers.

Among these cancers, the treatment of solid tumours and the treatment of cancers that are resistant to cytotoxic agents are of interest.

Among these cancers, the treatment of breast cancer, stomach cancer, cancer of the ovaries, cancer of the colon, lung cancer, brain cancer, cancer of the larynx, cancer of the lymphatic system, cancer of the genito-urinary tract including the bladder and the prostate, bone cancer and cancer of the pancreas, and most particularly treatment of breast cancer, cancer of the colon or lung cancer, are of interest.

A subject of the present invention is also the use of the products of formula (I) as defined above, for the preparation of medicinal products for cancer chemotherapy.

Such medicinal products intended for cancer chemotherapy may be used alone or in combination.

The products of the present patent application may especially be administered alone or in combination with chemotherapy or radiotherapy or alternatively in combination, for example, with other therapeutic agents.

Such therapeutic agents may be commonly-used anti-tumoral agents.

As kinase inhibitors, mention may be made of butyrolactone, flavopiridol and 2-(2-hydroxyethylamino)-6-benzylamino-9-methylpurine, also known as olomucine.

A subject of the present invention is also compounds of formula (I) as defined above, as inhibitors of one or more protein kinases chosen from FDR, Fak, tie2, Aurora, AKT and IGF-1R.

A subject of the present invention is particularly the compounds of formula (I) as defined above, as KDR inhibitors.

A subject of the present invention is also the products of formula (I) as defined above, as tie2 inhibitors.

### Experimental Section

Methods A to D below were used to prepare the products of formula (I) described in the examples below.

Method A: Analysis by LC/MS

The LC/MS analyses were performed on an LCT Micromass machine connected to an HP 1100 machine. The abundance of the products was measured using an HP G1315A diode array detector over a wavelength range of 200-600 nm and a Sedex 65 light scattering detector. The mass spectra were acquired over a range from 180 to 800. The data were analysed using the Micromass MassLynx software. The separation was performed on a Hypersil BDS C18 3 μm (50×4.6 mm) column, eluting with a linear gradient of 5% to 90% acetonitrile containing 0.05% (v/v) of trifluoroacetic acid (TFA) in water containing 0.05% (v/v) of TFA, over 3.5 minutes at a flow rate of 1 ml/minute. The total analysis time, including the column reequilibration period, is 7 minutes.

Method B: Purification by LC/MS:

The products were purified by LC/MS using a Waters FractionsLynx system composed of a Waters 600 gradient pump, a Waters 515 regeneration pump, a Waters Reagent Manager dilution pump, a Waters 2700 auto-injector, two Rheodyne LabPro valves, a Water 996 diode array detector, a Waters ZMD mass spectrometer and a Gilson 204 fraction collector. The system was controlled by the Waters FractionLynx software. The separation was performed alternately on two Waters Symmetry (C18, 5 μM, 19×50 mm, catalogue reference 186000210) columns, one column undergoing regeneration with a 95/5 (v/v) water/acetonitrile mixture containing 0.07% (v/v) of trifluoroacetic acid, while the other column is being used for separation. The columns were eluted using a linear gradient of 5% to 95% acetonitrile containing 0.07% (v/v) of trifluoroacetic acid in water containing 0.07% (v/v) of trifluoroacetic acid, at a flow rate of 10 ml/minute. On leaving the separation column, one thousandth of the effluent is separated out using an LC Packing Accurate machine, diluted with methanol at a flow rate of 0.5 ml/minute and conveyed to the detectors, in a proportion of 75% to the diode array detector and the remaining 25% to the mass spectrometer. The rest of the effluent (999/1000) is conveyed to the fraction collector, where the flow is discarded if the mass of the expected product is not detected by the FractionLynx software. The molecular formulae of the expected products are supplied to the FractionLynx software, which triggers the collection of the product when the mass signal detected corresponds to the [M+H]⁺ ion and/or to the [M+Na]⁺ ion. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, collection is also triggered when the mass signal of the [M+2H]⁺⁺ and/or [M+Na+H]⁺⁺ ion is detected.

Method C: EI Analysis

The mass spectra were acquired by electron impact (70 eV) on a Finnigan SSQ 7000 spectrometer.

Method D: NMR Analysis

The NMR spectra were acquired on Bruker Avance 300 and Bruker Avance DRX 400 spectrometers.

The present invention relates most particularly to the products of formula (I) represented in Table I below, and which constitute Examples 1 to 22 of the present invention.

The 22 products of formula (I) according to the present invention, the formulae of which are given in Table I, were prepared as indicated below.

These 22 products more specifically illustrate the present invention, without, however, limiting it.

In particular, the products of Examples 1 to 18 described below, in which the indazole radical is replaced with a pyrazole radical, may be prepared as indicated in Examples 19 to 22 of the present invention and form part of the present invention.

### EXAMPLE 1

### Preparation of 3-(5-cyanoindol-2-yl)indazole

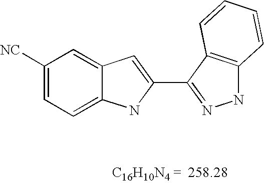

Step 1: N-Boc-5-cyanoindole

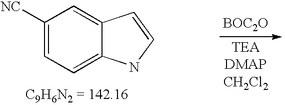

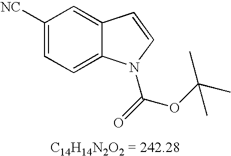

| | | |
|---|---|---|
| 5-Cyanoindole | | 1.0079 g = 7.09 mmol |
| BOC₂O | 1.2 eq | 8.51 mmol = 1.86 g |
| MW = 218.25 | | |
| Triethylamine (TEA) | | 2 ml |
| MW = 101.19 | | |
| d = 0.73 | | |
| 4-Dimethylaminopyridine (DMAP) | 10% | 0.709 mmol—87 mg |
| MW = 122.17 | | |
| CH₂Cl₂ | | 20 ml |

After introducing 5-cyanoindole, Boc2O, dichloromethane and DMAP into a 100 ml round-bottomed flask, the reaction medium is stirred at 0° C. under nitrogen for 2 hours. After disappearance of the starting cyanoindole, the reaction medium is poured into water and extracted with EtOAc. After drying and evaporating off the solvent, 1.7191 g of N-Boc-5-cyanoindole are obtained in the form of a yellowish powder.

Rf (silica)=0.61; 7/3 cyclohexane/EtOAc.

LC/MS m/z=242.

Step 2: N-Boc-5-cyanoindole-2-boronic acid

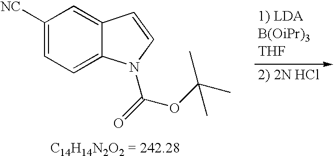

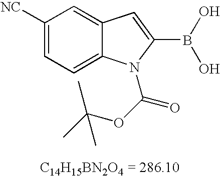

| | | |
|---|---|---|
| N-Boc-5-cyanoindole | | 1.0025 g = 4.14 mmol |
| Triisopropyl borate | 3 eq | 0.012 mol = 2.26 g |
| C₉H₂₁BO₃ = 188.07 | | 2.79 ml |
| d = 0.81 | | |
| Lithium diisopropylamide | 2.5 eq | 0.010 mol—6.9 ml |
| 1.5 M tetrahydrofuran/ | | |
| cyclohexane complex | | |
| (Aldrich) | | |
| THF | | 10 ml |
| 2N HCl | | |

The indole derivative in the THF is introduced into a 50 ml round-bottomed flask. The borate is added at room temperature under nitrogen, followed by dropwise addition over 20 minutes, at 0° C. under nitrogen, of the LDA. The mixture is stirred at 0° C. for 2 hours. The reaction medium is neutralized with 2N HCl and extracted with EtOAc. After drying and evaporating off the solvent, 829.3 mg of a brown foam containing 60% of the expected product, N-Boc-5-cyanoindole-2-boronic acid, and 40% of its Boc-free analogue, are obtained.

This product is used without further purification for the coupling in Step 3.

Step 3: 3-(5-Cyanoindol-2-yl)indazole

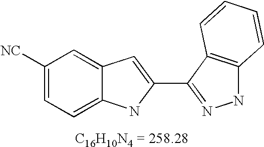

| | | |
|---|---|---|
| N-Boc-3-iodoindazole | | 503 mg—1.46 mmol |
| N-Boc-5-cyanoindole- | 1.5 eq | 2.19 mmol—627 mg |
| 2-boronic acid | | |
| C₁₄H₁₅BN₂O₃ = 286.10 | | |
| Palladium tetrakis- | 0.025 eq | 0.037 mmol—42 mg |
| triphenylphosphine | | |
| C₇₂H₆₀P₄Pd = 1155.58 | | |
| Saturated aqueous | | 1 ml |
| NaHCO₃ | | |
| DMF | | 10 ml |
| CH₂Cl₂ | | 10 ml |

The N-Boc-3-iodoindazole dissolved in the DMF is placed in a 50 ml round-bottomed flask. The N-Boc-5-cyanoindole-2-boronic acid, the NaHCO₃ solution and the Pd(PPh₃)₄ catalyst are then added, after which the reaction mixture is refluxed for 1 hour 30 minutes and poured into water, and the precipitate formed is filtered off. 792 mg of a mixture are thus obtained, which product is purified by chromatography on a column of Si60 silica (100 parts), eluting with: 95/5, 90/10, 80/20, 70/30 cyclohexane/EtOAc by volume.

224.9 mg of 3-(5-cyanoindol-2-yl)indazole are thus obtained in the form of a yellowish powder.

Rf (silica)=0.44; 95/5 CH₂Cl₂/MeOH.

LC/MS m/z=258.

### EXAMPLE 8

### 3-(5-Carboxyindol-2-yl)indazole

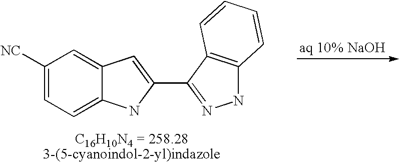

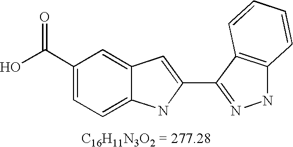

| | |
|---|---|
| 3-(5-Cyanoindol-2-yl) indazole | 170 mg—0.66 mmol |
| 10% NaOH | 10 ml |

The 3-(5-cyanoindol-2-yl)indazole and the aqueous 10% sodium hydroxide are introduced into a 30 ml round-bottomed flask and the reaction mixture is then refluxed for 2 hours. The reaction mixture is acidified with acetic acid and the precipitate formed is then filtered off. After drying, 131.8 mg of a yellow powder corresponding to the pure acid 3-(5-carboxyindol-2-yl)indazole are obtained.

Rf (silica)=0.44; 90/10 CH₂Cl₂/MeOH.

LC/MS retention time=3.90 minutes; m/z=278.

### EXAMPLE 9

### 3-(5-(N-(2-Chloropyrid-5-yl)methyl)carboxamideindol-2-yl)indazole

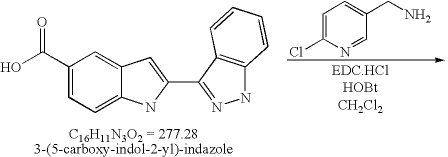

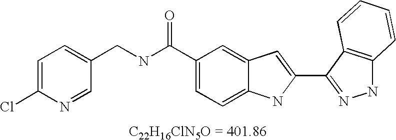

| | | |
|---|---|---|
| 3-(5-carboxy-indol-2-yl)-indazole | | 131.8 mg—0.47 mmol |
| 3-(5-Carboxyindol-2- | | |
| yl) indazole | | |
| 5-(Aminomethyl)-2- | 1.2 eq | 0.57 mmol—81 mg |
| chloropyridine | | |
| C₆H₇ClN₂ = 142.59 | | |
| N-(3-Dimethylamino- | 1.5 eq | 0.71 mmol—135 mg |
| propyl)-N′-ethyl- | | |
| carbodiimide·HCl | | |
| (EDCl) | | |
| MW = 191.71 | | |
| 1-Hydroxybenzo- | 1.5 eq | 0.71 mmol—95 mg |
| triazole hydrate | | |
| (HOBT) | | |
| C₆H₅N₃·xH₂O = 135.12 | | 10 ml |
| CH₂Cl₂ | | |

The acid 3-(5-carboxyindol-2-yl)indazole and the 5-(aminomethyl)-2-chloropyridine dissolved in 5 ml of CH₂Cl₂ are introduced into a 30 ml round-bottomed flask. The EDC and the HOBt dissolved in 5 ml of CH₂Cl₂ are then added at room temperature under nitrogen. The reaction mixture is stirred at room temperature under nitrogen for 24 hours. A sufficient amount of DMF to fully dissolve the reaction medium is added. A further 0.355 mmol of the reagents EDCI and HOBt are then added. The reaction medium is stirred at room temperature for 5 hours and then poured into water and extracted with EtOAc. After drying and concentrating, 268.5 mg of a yellow oil are thus obtained, which product is purified by chromatography on silica (Biotage), eluting with a 99.5/0.5, 98/2, 95/5, 91/10 CH₂Cl₂/MeOH mixture by volume. 23.9 mg of 3-(5-(N-(2-chloropyrid-5-yl)methyl)carboxamideindol-2-yl)indazole are thus obtained in the form of a beige-coloured powder. 118.6 mg of a mixture are also obtained, which mixture is repurified by chromatography on a column of 60H silica (12 g), eluting with 99/1, 98/2 CH₂Cl₂/MeOH by volume. Two fractions of comparable purity of 3-(5-(N-(2-chloropyrid-5-yl)methyl)carboxamideindol-2-yl)indazole are thus obtained (41.8 mg and 49.6 mg, respectively) in the form of whitish powders.

### EXAMPLE 11

### 3-(5-Aminomethylindol-2-yl)indazole

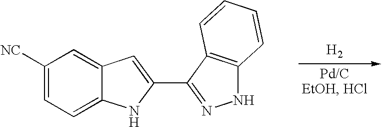

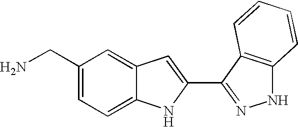

| Reagents: | | |
|---|---|---|
| 3-(5-Cyanoindol-2-yl) | | 83 mg—0.32 mmol |
| indazole | | |
| Palladium-on-charcoal | 20% | 72 mg—0.064 mmol |
| (at 9.5%) | | |
| 1N HCl | 2 eq | 0.64 ml |
| EtOH | 5 ml | |
| H₂ | | |

Procedure:

The 3-(5-cyanoindol-2-yl)indazole, the palladium-on-charcoal, the ethanol and the 1N hydrochloric acid are placed in a 30 ml round-bottomed flask equipped with a magnetic stirrer and the mixture is stirred under an H₂ atmosphere (rubber balloon) for 24 hours at 20° C. A further 70 mg of palladium-on-charcoal and 0.3 ml of 1N HCl are added and the mixture is then stirred under an H₂ atmosphere for 3 hours: the reaction is complete. The reaction mixture is filtered through clarcel and the solvent is concentrated under reduced pressure to a yellow powder. The crude product is purified by chromatography on silica (Biotage) with a 95/5 to 80/20 CH₂Cl₂/B elution gradient, the solvent B being a 38/17/2 CH₂Cl₂/CH₃OH/NH₄OH ternary mixture.

44.3 mg of 3-(5-aminomethylindol-2-yl)indazole are thus isolated in the form of a yellowish powder, i.e. a 53% yield.

Rf=0.37 (CH₂Cl₂/B 50/50).

EI-MS: 262(+)=M(+).

The products of Examples 2 to 7, 10 and 13 may be prepared as described for Example 1, by replacing in Stage 1 of Example 1 the 5-cyanoindole with the following starting materials, respectively:

indole

5-ethoxycarbonylindole

5-(N,N-diisopropyl)carboxamideindole

5-methylindole

5-chloroindole

6-methylindole

5-(morpholinoethyloxy)indole

6-methoxycarbonylindole

The process is then performed in the same manner as in Stages 2 and 3 of Example 1, starting with the products obtained in Stage 1, respectively, and the expected products of Examples 2 to 7, 10 and 13 are thus obtained.

The product of Example 15 is prepared as described for Example 8, working according to the same procedure, starting with the product of Example 13 instead of the product of Example 1.

The product of Example 14 is prepared as described for Example 9, working according to the same procedure, starting with the product of Example 11 instead of the product of Example 8.

The product of Examples 12 is prepared as described for Example 9, working according to the same procedure, starting with the product of Example 8.

The products of Examples 16, 17 and 18 are prepared as described for Example 9, working according to the same procedure, starting with the product of Example 15 instead of the product of Example 8.

### EXAMPLE 19

### 4-Amino-3-(indol-2-yl)pyrazole

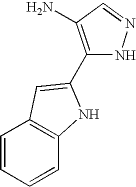

4-Amino-3-(indol-2-yl)pyrazole may be Prepared in the following Manner:

761.9 mg of N-Boc-indolyl-2-boronic acid, an aqueous solution of 122.6 mg of sodium bicarbonate and 421.6 mg of tetrakis(triphenylphosphine)palladium are successively added to a solution of 280.1 mg of 3-bromo-4-nitropyrazole in 10 ml of anhydrous dimethylformamide. The reaction mixture is stirred under an argon atmosphere at a temperature in the region of 135° C. for about 20 hours. After evaporating off the solvent under reduced pressure, the greyish solid obtained is taken up in methanol and filtered off over Celite. The filtrate is purified by passing it through a cartridge of SPE (SCX phase, washing with methanol and then extraction of the product with a 2N ammoniacal solution in methanol). After evaporating off the solvent, the brown oil obtained (98.6 mg) is purified by chromatography on silica (dichloromethane/methanol elution gradient from t=0 0% methanol to t=30 min 10% methanol). The fractions containing the desired product are combined and concentrated under reduced pressure. 25.3 mg of a product are thus obtained, which product is purified by preparative LC/MS (method B). After passage through SPE (SCX phase), 3.9 mg of 4-amino-3-(indol-2-yl)pyrazole are thus obtained in the form of a solid, the characteristics of which are as follows:

LC/MS retention time=2.16 minutes; m/z=199.2 The 3-bromo-4-nitropyrazole may be prepared from 3-bromopyrazole by nitration according to the conditions described for 3-chloropyrazole in patent U.S. Pat. No. 3,869,274.

### EXAMPLE 20

### 3-[5-(1H-indol-2-yl)-2H-pyrazol-3-yloxymethyl]phenol

3-[5-(1H-Indol-2-yl)-2H-pyrazol-3-yloxymethyl]phenol may be Prepared According to the Following Manner:

A solution of 3-[5-(1-phenylsulphonyl-1H-indol-2-yl)-2H-pyrazol-3-yloxymethyl]phenol in methanolic potassium hydroxide (2N solution of KOH in methanol) is maintained at reflux until the starting material has disappeared. After cooling to a temperature in the region of 20° C., the reaction medium is neutralized by slow addition of concentrated hydrochloric acid, and then extracted with ethyl acetate. The combined organic phases are dried over magnesium sulphate, filtered through a sinter funnel, concentrated under reduced pressure and purified by preparative LC/MS (method B). The fractions containing the desired product are combined and concentrated to dryness under reduced pressure. After treatment by SPE (SCX phase), 3-[5-(indol-2-yl)-2H-pyrazol-3-yloxymethyl]phenol is thus obtained.

3-(5-(1-Phenylsulphonyl-1H-indol-2-yl)-2H-pyrazol-3-yloxymethyl]phenol may be Prepared in the Following Manner:

Caesium carbonate (1.2 equivalents) is added to a solution of 5-(1-(phenylsulphonyl)-1H-indole-2-yl)pyrazol-3-ol (1 equivalent) in dimethylformamide, at a temperature in the region of 20° C., followed by slow portionwise addition of a solution of 1-bromomethyl-(3-benzoyloxy)phenyl (1 equivalent) in dimethylformamide. The reaction medium is filtered through Celite, concentrated under reduced pressure and purified by preparative LC/MS (method B). The fractions containing the desired product are combined and concentrated to dryness under reduced pressure. After treatment by SPE (SCX phase), 3-[5-(1-phenylsulphonyl-1H-indol-2-yl)-2H-pyrazol-3-yloxymethyl]phenol is thus obtained.

5-(1-(Phenylsulphonyl)-1H-indol-2-yl)pyrazol-3-ol may be Prepared in the Following Manner:

5.43 ml of hydrazine hydrate are added to a suspension of 20 g of methyl 3-(1-(phenylsulphonyl)-1H-indol-2-yl)-3-oxopropionate in 200 ml of ethanol, at a temperature in the region of 20° C. After 4 hours at a temperature in the region of 20° C., 1.63 ml of hydrazine hydrate are added and the solution is refluxed for 1 hour and then left at a temperature close to 20° C. for 16 hours. The black solution obtained is concentrated to dryness under reduced pressure, at a temperature in the region of 40° C., and the solid residue is then washed twice with a CH₂Cl₂/methanol/NH₃—H₂O mixture (12/3/0.5 by volume). After filtering off the solid residue, the filtrate is concentrated to dryness under reduced pressure at a temperature in the region of 40° C. 19 g of a solid red deposit are thus obtained, and are taken up in 50 ml of a CH₂Cl₂/methanol/NH₃—H₂O mixture (12/3/0.5 by volume), filtered through a sinter funnel and rinsed with a CH₂Cl₂/methanol/NH₃—H₂O mixture (12/3/0.5 by volume). The red filtration liquors are concentrated to dryness under reduced pressure, at a temperature in the region of 40° C., and purified by chromatography on a column of silica (diameter 10 cm; 1000 g of 70-200 μm silica; 1000 ml fractions; eluent: CH₂Cl₂/methanol/NH₃—H₂O (12/3/0.5 by volume)). 1.47 g of 5-(1-phenylsulphonyl)-1H-indol-2-yl)pyrazol-3-ol are thus obtained in the form of a beige-coloured powder (Rf=0.25; SiO₂; eluent: CH₂Cl₂/methanol/NH₃—H₂O (12/3/0.5 by volume)).

Methyl 3-(1-(phenylsulphonyl)-1H-indol-2-yl)-3-oxo-propionate may be Prepared in the Following Manner:

22.8 g of Meldrum's acid and 38.7 g of N,N-dimethylamino-4-pyridine are successively added to 500 ml of chloroform, at a temperature in the region of 20° C. 50.6 g of 1-(phenylsulphonyl)indole-2-carboxylic acid chloride dissolved in 150 ml of chloroform are added, over 30 minutes, to the colourless solution thus obtained, maintained at a temperature in the region of 0° C. The brown solution obtained is stirred for 1 hour at a temperature in the region of 0° C. and the temperature is then allowed to rise to about 20° C. The solution is brought to a pH close to 2 by addition of aqueous 2 N hydrochloric acid solution (about 80 ml) and then diluted with 250 ml of water. After separation of the phases by settling, the organic phase is washed with twice 250 ml of water and then with 250 ml of saturated aqueous sodium chloride solution, dried over magnesium sulphate, filtered on paper and concentrated to dryness under reduced pressure at a temperature in the region of 400C. The viscous brown oil obtained is taken up in 830 ml of methanol and the solution thus obtained is refluxed for 2 hours. After cooling to a temperature in the region of 20° C., the reaction mixture is concentrated to dryness under reduced pressure at a temperature in the region of 40° C. and then purified by chromatography on a column of silica (diameter 10 cm; silica height 33 cm; 100 ml fractions; eluent: dichloromethane). 21 g of 3-(1-(phenylsulphonyl)-1H-indol-2-yl)-3-oxopropionate are thus obtained in the form of a cream-coloured solid (Rf=0.39; SiO₂; CH₂Cl₂=100).

1-(Phenylsulphonyl)indole-2-carboxylic Acid Chloride may be Prepared in the Following Manner:

310 ml of thionyl chloride are added slowly, at a temperature in the region of 20° C., to 47.7 g of 1-(phenylsulphonyl)indole-2-carboxylic acid. The brown suspension obtained is brought slowly to the reflux temperature. This heating is maintained for 3 hours. After cooling to a temperature in the region of 20° C., the reaction mixture is concentrated to dryness under reduced pressure, at a temperature in the region of 40° C. The brown residue obtained is taken up 3 times with 300 ml of anhydrous cyclohexane and concentrated to dryness under reduced pressure, at a temperature in the region of 40° C. After drying under reduced pressure, 50 g of 1-(phenylsulphonyl)indole-2-carboxylic acid are obtained in the form of a brown solid, which is used without further purification.

1-(Phenylsulphonyl)indole-2-carboxylic Acid may be Prepared in the Following Manner:

160 ml of a 1.6 M solution of n-butyllithium in hexane are added dropwise to a solution of 36 ml of diisopropylamine in 300 ml of tetrahydrofuran, under an inert atmosphere of argon, maintained at a temperature in the region of −70° C. with an acetone/cardice bath. The acetone/cardice bath is removed and replaced with a water/ice bath. A solution of 57.7 g of 1-(phenylsulphonyl)indole in 400 ml of tetrahydrofuran is then added dropwise at a temperature in the region of 0° C. After 30 minutes at a temperature in the region of 0° C., the solution is cooled to a temperature in the region of −0° C. About 100 g of cardice are added slowly to the bright orange solution obtained, and the temperature of the solution is then allowed to return to a temperature in the region of 12° C. The reaction medium is concentrated to ¾ under reduced pressure at a temperature in the region of 40° C. The dark orange syrup obtained is taken up in 50 ml of water and extracted with twice 250 ml of ethyl ether. The aqueous phase is acidified to a pH in the region of 2 by adding 2 N hydrochloric acid, and then extracted 4 times with ethyl ether. The organic phases are combined, washed with twice 200 ml of water, dried over magnesium sulphate containing black 3S, filtered through paper and then concentrated to dryness under reduced pressure at a temperature in the region of 40° C. 45.6 g of 1-(phenylsulphonyl)indole-2-carboxylic acid are thus obtained in the form of a pale beige-coloured solid (Rf=0.39; SiO₂; CH₂Cl₂/methanol/NH₃-H₂O=12/3/0.5 by volume).

### EXAMPLE 21

### N-{3-[5-(indol-2-yl)-2H-pyrazol-3-yloxymethyl]phenyl}acetamide

N-{3-[5-(Indol-2-yl)-2H-pyrazol-3-yloxymethyl]phenyl}acetamide may be prepared according to a method similar to the one used in Example 20 for the preparation of 3-[5-(1H-indol-2-yl)-2H-pyrazol-3-yloxyethyl]phenol, from N-{3-[5-(1-phenylsulphonyl-1H-indol-2-yl)-2H-pyrazol-3-yloxymethyl]phenyl}acetamide.

N-{3-[5-(1-Phenylsulphonyl-1H-indol-2-yl)-2H-pyrazol-3-yloxymethyl]phenyl}acetamide may be prepared according to a method similar to the one used in Example 20 for the preparation of 3-[5-(1-phenylsulphonyl-1H-indol-2-yl)-2H-pyrazol-3-yloxymethyl]phenol, from 5-(1-(phenylsulphonyl)-1H-indol-2-yl)pyrazol-3-ol (1 equivalent) and 1-bromomethyl-(3-acetylamino)phenyl (1 equivalent).

### EXAMPLE 22

### 2-[5-(3-fluorobenzyloxy)-1H-pyrazol-3-yl]-1H-indole

2-[5-(3-Fluorobenzyloxy)-1H-pyrazol-3-yl]-1H-indole may be prepared according to a method similar to the one used in Example 20 for the preparation of 3-[5-(1H-indol-2-yl)-2H-pyrazol-3-yloxymethyl]phenol, from 1-phenylsulphonyl-2-[5-(3-fluorobenzyloxy)-1H-pyrazol-3-yl]-1H-indole.

1-Phenylsulphonyl-2-[5-(3-fluorobenzyloxy)-1H-pyrazol-3-yl]-1H-indole may be prepared according to a method similar to the one used in Example 20 for the preparation of 3-[5-(1-phenylsulphonyl-1H-indol-2-yl)-2H-pyrazol-3-yloxymethyl]phenol, from 5-(1-(phenylsulphonyl)-1H-indol-2-yl)pyrazol-3-ol (1 equivalent) and 1-bromomethyl-3-fluorophenyl (1 equivalent).

### EXAMPLE 23

### Pharmaceutical Composition

Tablets corresponding to the formula below were prepared:

| | |
|---|---|
| Product of Example 9 | 0.2 g |
| Excipient for a finished tablet containing | 1 g |
| (details of the excipient: lactose, talc, starch, | |
| magnesium stearate). | |

### EXAMPLE 24

### Pharmaceutical Composition

Tablets corresponding to the formula below were prepared:

| | |
|---|---|
| Product of Example 16 | 0.2 g |
| Excipient for a finished tablet containing | 1 g |
| (details of the excipient: lactose, talc, starch, | |
| magnesium stearate). | |

Examples 9 and 16 are given as examples of a pharmaceutical preparation, this preparation possibly being prepared, if desired, with other products given as examples in the present patent application.

Table I of the 22 products illustrated

| Examples | Structure | Nomenclature |
|---|---|---|
| 1 | 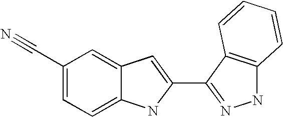 | 3-(5-cyanoindol-2-yl)indazole |
| 2 | 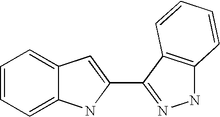 | 3-(indol-2-yl)indazole |
| 3 | 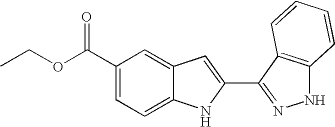 | 3-(5-ethoxycarbonylindol-2-yl)- indazole |
| 4 | 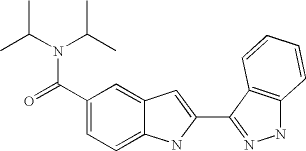 | 3-(5-(N,N-diisopropyl)carboxamide indol-2-yl)indazole |
| 5 | 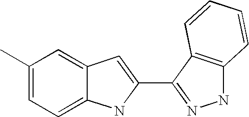 | 3-(5-methylindol-2-yl)indazole |
| 6 | 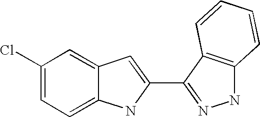 | 3-(5-chloroindol-2-yl)indazole |
| 7 | 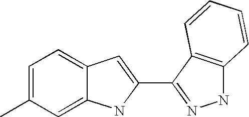 | 3-(6-methylindol-2-yl)indazole |
| 8 | 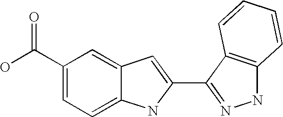 | 3-(5-carboxyindol-2-yl)indazole |
| 9 | 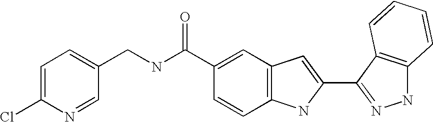 | 3-(5-(N-(2-chloropyrid-5-yl)methyl)- carboxamideindol-2-yl)indazole |
| 10 | 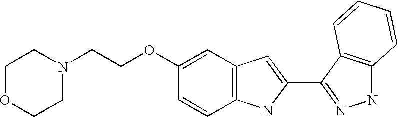 | 3-(5-(morpholinoethyloxy)indol-2-yl)- indazole |
| 11 | 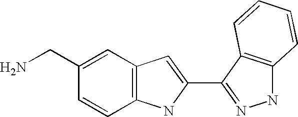 | 3-(5-aminomethylindol-2-yl)indazole |
| 12 | 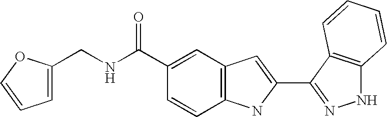 | 3-(5-(N-((2-furyl)methyl))- carboxamideindol-2-yl)indazole |
| 13 | 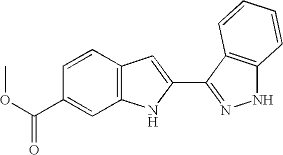 | 3-(6-methoxycarbonylindol-2-yl)- indazole |
| 14 | 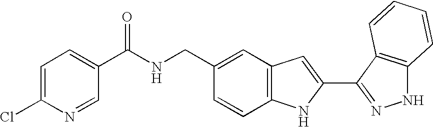 | 3-(5-((2-chloropyrid-5-yl)- carboxamido)methylene)indol-2-yl)- indazole |
| 15 | 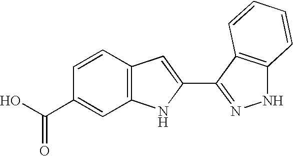 | 3-(6-carboxyindol-2-yl)indazole |
| 16 | 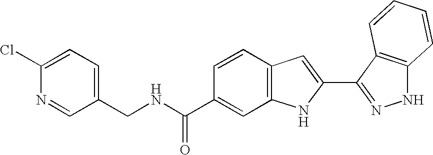 | 3-(6-(N-(2-chloropyrid-5-yl)methyl)- carboxamideindol-2-yl)indazole |
| 17 | 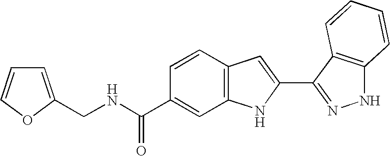 | 3-(6-(N-((2-furyl)methyl))- carboxamideindol-2-yl)indazole |
| 18 | 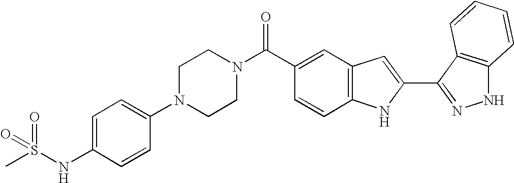 | 3-(5-(N-(4-methylsulphonamide- phenyl)pipérazinocarboxamide)- indol-2-yl)indazole |
| 19 | 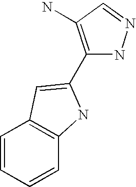 | 4-amino-3-(indol-2-yl)pyrazole |
| 20 | 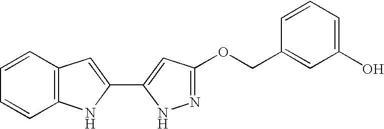 | 3-[5-(1H-indol-2-yl)-2H-pyrazol-3- yloxymethyl]-phenol |
| 21 | 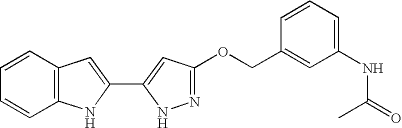 | N-{3-[5-(indol-2-yl)-2H-pyrazol-3- yloxymethyl]phenyl}acetamide |
| 22 | 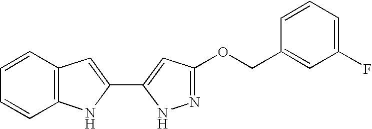 | 2-[5-(3-fluorobenzyloxy)-1H-pyrazol 3-yl]-1H-indole |

1) A compound of formula (I): 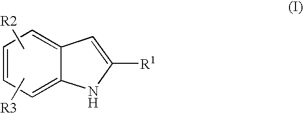
in which:
R1 is a pyrazolyl or indazolyl radical, said pyrazolyl or indazolyl radical optionally being substituted with one or more radicals selected from the group consisting of halogen, hydroxyl, nitro, cyano, R4, OR4, SR4, —COR4, —OC(═O)R4, —C(═O)OR4, free —C(═O)OH or a salt thereof, —N(R5)C(═O)R4, —N(R5)C(═O)OR4, —S(O)nR4, —S(O)nOR4, —N(R5)SO2R4, —S(O)nR4, —NY1Y2, —C(═O)NY1Y2, —OC(═O)NY1Y2, —N(R5)C(═O)NY1Y2, —S(O)nNY1Y2 and thienyl radicals, which radicals are optionally substituted,
R2 and R3 are such that:
either R2 and R3, which may be identical or different, are selected from a hydrogen atom, halogen atoms and hydroxyl, nitro, cyano, R4, —OR4, —COR4, —OC(═O)R4, —C(═O)OR4, —C(═O)OH, —N(R5)C(═O)R4, —N(R5)C(═O)OR4, —S(O)nR4, —S(O)nOR4, —N(R5)SO2R4, —NY1Y2, —C(═O)NY1Y2, —N(R5)C(═O)NY1Y2, —S(O)nNY1Y2 and —OC(═O)NY1Y2 radicals,
or R2 and R3, together with the phenyl residue of the indole radical, form a 4- to 6-membered carbon-based ring optionally containing one or more identical or different hetero atoms selected from O, N and S, this ring optionally being substituted,
R4 is selected from alkyl, alk-NY1Y2, alk-CO—NY1Y2, alkenyl, alkynyl, cycloalkyl, aryl, heteroaryl, cycloalkylalkyl, heterocycloalkyl, heteroarylalkyl and arylalkyl, all these radicals optionally being substituted,
R5 is selected from hydrogen, alkyl, alkenyl, cycloalkyl, heterocycloalkyl, aryl, heteroaryl, arylalkyl, cycloalkylalkyl, heteroarylalkyl and heterocycloalkylalkyl radicals, which are optionally substituted,
Y1 and Y2 are such that:
either Y1 and Y2, which may be identical or different, are selected from H, alkyl, alkenyl, cycloalkyl, heterocycloalkyl, heterocycloalkylalkyl, aryl, arylalkyl, arylcarboxyl, heteroaryl, heteroarylalkyl and heteroarylcarboxyl, all these radicals being optionally substituted,
or Y1 and Y2, together with the nitrogen atom to which they are attached, form an optionally substituted cyclic amino radical,
all the alkyl, alkenyl, alkynyl, cycloalkyl, cycloalkylalkyl, heterocycloalkyl, heterocycloalkylalkyl, aryl, aryloxy, arylalkyl, arylcarboxyl, heteroaryl, heteroarylalkyl and heteroarylcarboxyl radicals being optionally substituted with one or more radicals selected from halogen atoms and hydroxyl, alkoxy, alkyl, hydroxyalkyl, carboxyalkyl, cyano, nitro, trifluoromethyl, trifluoromethoxy radicals, a carboxyl radical that is free, salified or esterified with an optionally substituted alkyl radical, —Nalk-COalk, —NH—COalk, S(O)n-alk, NH-S(O)n-alkyl, —NHCO—NY3Y4, —C(═O)—NY3Y4 and S(O)n-NY3Y4, aryl, arylalkoxy, aryloxy, aryloxyalkyl, heteroaryl and heterocycloalkyl radicals, which are optionally substituted,
Y3 and Y4, which may be identical or different, are selected from hydrogen, alkyl and aryl, which are optionally substituted,
these latter alkyl (alk), heterocycloalkyl, aryl and heteroaryl radicals themselves optionally being substituted with one or more radicals selected from halogen atoms and alkyl, free, salified or esterified carboxyl, amino, alkylamino, dialkylamino and phenylamino, hydroxyl, alkoxy and NHCO alkyl radicals,
all the phenyl radicals also being optionally substituted with a dioxole radical,
n is an integer from 0 to 2,
alk is alkyl of 1 to 6 carbon atoms,
with the exception of the products defined below in i), ii) and iii):
i) compounds of formula (I) in which R2 and R3 both represent a nitro radical, the other substituents of said compounds of formula (I) having the meanings indicated above,
ii) compounds of formula (I) as defined in formula (F): 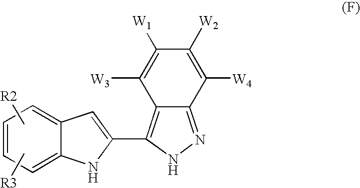
in which R2 and R3 are as defined above and W3 and W4 both are hydrogen, then:
either W1 is hydrogen and W2 is not aryl, heteroaryl or Y—X, Y being selected from O, S, C═CH2, C═O, S═O, SO2, alkylidene, NH and N(C1-C8)alkyl and X being selected from aryl, heteroaryl, NH(alkyl), NHcycloalkyl, NH(heterocycloalkyl), NH(aryl), NH(heteroaryl), NH(alkoxy) and NH(dialkylamide),
or W2 is hydrogen and W1 is not alkyl, alkenyl, aryl, heteroaryl, carbocycle or heterocycle,
iii) products of formula (I) as defined in formula (FF): 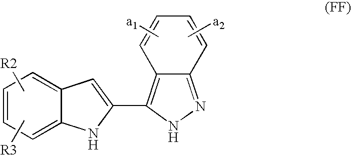
in which
R2 and R3, which may be identical or different, are selected from: hydrogen, COOalkyl, COOaryl, COOalkenyl, COOalkynyl, CO2H, halogen, OH, O-perfluoroalkyl, CONR7R8, CN, COOcycloalkyl, COOheterocyclyl, SO2NR7R8, SO2alkyl, which are optionally substituted,
it being understood that one of R2 and R3 is not hydrogen,
and a1 and a2 are selected from hydrogen, COOalkyl, COOaryl, COOalkenyl, COOalkynyl, CO2H, halogen, OH, O-perfluoroalkyl, CONR7R8, CN, COOcycloalkyl, COOheterocyclyl, SO2NR7R8 and SO2alkyl, which are optionally substituted,
said compounds of formula (I) being in all the possible racemic, enantiomeric and diastereoisomeric isomer forms, and also the addition salts with mineral and organic acids or with mineral bases.

2) A compound of formula (I) as defined in claim 1 in which R1 is a pyrazolyl or indazolyl radical, these pyrazolyl or indazolyl radicals optionally being substituted with one or more radicals selected from halogen, hydroxyl, nitro, cyano, R4, OR4, SR4, —COR4, —OC(═O)R4, —C(═O)OR4, free or salified —C(═O)OH, —N(R5)C(═O)R4, —N(R5)C(═O)OR4, —S(O)nR4, —S(O)nOR4, —N(R5)SO2R4, —OS(O)nR4, —NY1Y2, —C(═O)NY1Y2, —OC(═O)NY1Y2, —N(R5)C(═O)NY1Y2, —S(O)nNY1Y2 and thienyl radicals, which are optionally substituted,
R2 and R3 are such that:
either R2 and R3, which may be identical or different, are selected from hydrogen, halogen, hydroxyl, nitro, cyano, R4, —OR4, —COR4, —OC(═O)R4, —C(═O)OR4, —C(═O)OH, —N(R5)C(═O)R4, —N(R5)C(═O)OR4, —S(O)nR4, —S(O)nOR4, —N(R5)SO2R4, —NY1Y2, —C(═O)NY1Y2, —N(R5)C(═O)NY1Y2, —S(O)nNY1Y2 and —OC(═O)NY1Y2 radicals,
or R2 and R3, together with the phenyl residue of the indole radical, form a 4- to 6-membered carbon-based ring optionally containing one or more identical or different hetero atoms selected from O, N and S, this ring optionally being substituted,
R4 is selected from alkyl, alk-NY1Y2, alk-CO—NY1Y2, alkenyl, alkynyl, cycloalkyl, aryl, heteroaryl, cycloalkylalkyl, heterocycloalkyl, heteroarylalkyl and arylalkyl, all these radicals optionally being substituted,
R5 is selected from hydrogen, alkyl, alkenyl, cycloalkyl, heterocycloalkyl, aryl, heteroaryl, arylalkyl, cycloalkylalkyl, heteroarylalkyl and heterocycloalkylalkyl, which are optionally substituted,
Y1 and Y2 are such that:
either Y1 and Y2, which may be identical or different, are selected from H, alkyl, alkenyl, cycloalkyl, heterocycloalkyl, heterocycloalkylalkyl, aryl, arylalkyl, arylcarboxyl, heteroaryl, heteroarylalkyl and heteroarylcarboxyl, all these radicals being optionally substituted,
or Y1 and Y2, together with the nitrogen atom to which they are attached, form an optionally substituted cyclic amino radical,
all the alkyl, alkenyl, alkynyl, cycloalkyl, cycloalkylalkyl, heterocycloalkyl, heterocycloalkylalkyl, aryl, aryloxy, arylalkyl, arylcarboxyl, heteroaryl, heteroarylalkyl and heteroarylcarboxyl radicals being optionally substituted with one or more radicals selected from halogen, hydroxyl, alkoxy, alkyl, hydroxyalkyl, carboxyalkyl, cyano, nitro, trifluoromethyl, trifluoromethoxy, a carboxyl radical that is free, salified or esterified with an optionally substituted alkyl radical, —Nalk-COalk, —NH—COalk, S(O)n-alk, NH—S(O)n-alkyl, —NHCO—NY3Y4, —C(═O)—NY3Y4 and S(O)n-NY3Y4, aryl, arylalkoxy, aryloxy, aryloxyalkyl, heteroaryl and heterocycloalkyl radicals, which are optionally substituted,
Y3 and Y4, which may be identical or different, are selected from hydrogen, alkyl and aryl, which are optionally substituted,
these latter alkyl (alk), heterocycloalkyl, aryl and heteroaryl radicals themselves optionally being substituted with one or more radicals selected from halogen atoms and alkyl, free, salified or esterified carboxyl, amino, alkylamino, dialkylamino and phenylamino radicals,
all the phenyl radicals also being optionally substituted with a dioxole radical,
n is an integer from 0 to 2,
alk is alkyl of 1 to 6 carbon atoms,
said compounds of formula (I) being in all the possible racemic, enantiomeric and diastereoisomeric isomer forms, and also the addition salts with mineral and organic acids or with mineral bases.

3) A compound of formula (I) as defined in claim 1, in which R1 is a pyrazolyl or indazolyl radical, these radicals being optionally substituted with one or more radicals selected from the group consisting of halogen, hydroxyl, nitro, cyano, R4, OR4, SR4, —COR4, —OC(═O)R4, —C(═O)OR4, free —C(═O)OH or a salt thereof, —N(R5)C(═O)R4, —N(R5)C(═O)OR4, —S(O)nR4,
—S(O)nOR4, —N(R5)SO2R4, —OS(O)nR4, —NY1Y2, —C(═O)NY1Y2, —OC(═O)NY1Y2, —N(R5)C(═O)NY1Y2, —S(O)nNY1Y2 and thienyl radicals, which radicals are optionally substituted,
R2 and R3, which may be identical or different, are selected from hydrogen, halogen, hydroxyl, an alkyl radical optionally substituted with NY1Y2, alkenyl, —OR4, —CO—R4, —O—COR4, —OS(O)nR4, —O(CH2)n-CO—R4, nitro, cyano, aryl, heteroaryl and aryloxy radicals, a carboxyl radical, which carboxyl radical is free, salified or esterified with an alkyl radical optionally substituted or amidated with a radical NY1Y2 such that either Y1 and Y2, which may be identical or different, are selected from H, alkyl, alkoxyalkyl, cycloalkyl, phenoxyalkyl, aryl, arylalkyl, cycloalkylalkyl, heterocycloalkylalkyl, heteroarylalkyl, arylcarboxyl and heteroarylcarboxyl, which are optionally substituted, or Y1 and Y2, together with the nitrogen atom to which they are attached, form an optionally substituted 5- or 6-membered cyclic radical,
it being understood that R2 and R3, which are consecutive may, together with the indole radical to which they are attached, form a 5- to 6-membered carbon-based ring containing one or more identical or different hetero atoms selected from O, N and S,
R4 is selected from alkyl, alkenyl, cycloalkyl, aryl, heteroaryl and cycloalkylalkyl, which are optionally substituted,
all the alkyl, alkenyl, aryl, heteroaryl, aryloxy, cycloalkyl and heterocycloalkyl radicals contained in the above radicals being optionally substituted with one or more radicals selected from halogen, hydroxyl, alkoxy, alkyl, hydroxyalkyl, carboxyalkyl, cyano, nitro, trifluoromethyl, trifluoromethoxy, phenyl, thienyl, phenoxy, phenoxyalkyl, phenylalkoxy, —NH2, —NH(alk), —N(alk)2, —NH—SO2-alkyl, —NH(phenyl) and —NH(phenylalkyl) radicals, a carboxyl radical which is free, salified or esterified with an optionally substituted alkyl radical, —C(═O)—NH2, —C(═O)—NH(alk), C(═O)—N(alk)2, —NH—COalk, —C(═O)alk, —N(H)C(═O)alk, S(O)n-alk, S(O)n-NH2, S(O)n-NH(alk) and S(O)n-N(alk)2 radicals,
all the alkyl, alkenyl, alkoxy and alkylthio radicals above being linear or branched and containing not more than 6 carbon atoms,
all the phenyl radicals of the above radicals also being optionally substituted with a dioxole radical and one or more halogen atoms,
n is an integer from 0 to 2,
the said compounds of formula (I) being in all the possible racemic, enantiomeric and diastereoisomeric isomer forms, and also the addition salts with mineral and organic acids or with mineral and organic bases of said compounds of formula (I).

4) A compound of formula (I) as defined in claim 1, in which R1 is a pyrazolyl or indazolyl radical optionally substituted with one or two radicals selected from halogen, OH, R4, OR4, SR4, —COR4, —O—COR4, —OS(O)nR4, NO2, CN, CF3, OCF3, NY1Y2, free or salified or esterified carboxyl, —C(═O)—NY1Y2, —N(R5)C(═O)NY1Y2, —NH—CO—R4, S(O)n-alk, S(O)n-NY1Y2, —NR5-C(═O)R4, —NR5-S(O)nR4, —NR5-C(═O)OH, —NR5-C(═O)OR4, —OC(═O)NY1Y2 and thienyl radicals, all these radicals being optionally substituted,
R2 and R3, which may be identical or different, are selected from hydrogen; halogen; hydroxyl; alkyl optionally substituted with NY1Y2; alkenyl; alkoxy; nitro; cyano; furyl; thienyl; benzothienyl; naphthyl; thianthrenyl; phenyl; phenoxy and carboxyl which is free, salified or esterified with an alkyl radical or amidated with NY1Y2,
it being understood that R2 and R3 may, together with the indole radical to which they are attached, form a 4,5-ethylenedioxybenzimidazole radical or a 4,5-methylenedioxybenzimidazole radical, which are optionally substituted,
NY1Y2 are such that either Y1 and Y2, which may be identical or different, are selected from hydrogen; alkyl; alkoxyalkyl; phenoxyalkyl; phenyl; phenylalkyl; phenylcarboxyl; naphthyl; naphthylalkyl; cycloalkylalkyl; cycloalkyl; furylalkyl; naphthylalkyl; thienylalkyl; piperidylalkyl; pyridylalkyl; benzothienylalkyl; pyrazolylalkyl; pyridylcarboxyl; dihydrobenzofuranalkyl; hexahydropyranalkyl; ethylenedioxyphenylalkyl; and benzimidazolylalkyl radicals; all these radicals being optionally substituted,
or Y1 and Y2, together with the nitrogen atom to which they are attached, form a pyrrolidinyl, pyrazolidinyl, pyrazolinyl, piperidyl, hexahydrofuran, morpholinyl or piperazinyl radical optionally substituted on the second nitrogen atom with an alkyl or phenyl radical, which alkyl and phenyl radicalsmay themselves be optionally substituted,
R4 is selected from alkyl, alkenyl, cycloalkyl, phenyl and cycloalkylalkyl, which are optionally substituted,
R5 is hydrogen, alkyl or phenyl, which are optionally substituted,
all the alkyl, alkenyl, phenyl, phenoxy, furyl, thienyl, piperidyl, pyridyl, pyrazolyl and benzimidazolyl radicals contained in the above radicals being optionally substituted with one or more radicals selected from halogen, hydroxyl, alkoxy, cyano, nitro, alkyl, hydroxyalkyl, carboxyalkyl, CF3, OCF3, NH2, NHalk, N(alk)2, NH(phenyl), NH(phenylalkyl), carboxyl which is free, salified or esterified with an alkyl radical, —C(═O)—NH2, —C(═O)—NH(alk), C(═O)—N(alk)2, NH—COalk, —C(═O)alk, S(O)n-alk, S(O)n-NH2, S(O)n-NH(alk), S(O)n-N(alk)2, thienyl, phenylalkyl, phenoxyalkyl, phenoxy, phenylalkoxy, morpholino, piperidyl and phenyl, in all these radicals the phenyl radical itself being optionally substituted with one or more radicals selected from halogen, cyano, CF3, OCF3, alkyl, phenyl-S(O)n-alk-phenyl, alkoxy, NH2, NHalk, N(alk)2, SO2NH2, SO2Nalk and SO2N(alk)2,
n is an integer from 0 to 2,
all the alkyl, alkenyl, alkoxy and alkylthio radicals above being linear or branched and containing not more than 6 carbon atoms,
all the phenyl radicals of the above radicals also being optionally substituted with a dioxole radical,
said compounds of formula (I) being in all the possible racemic, enantiomeric and diastereoisomeric isomer forms, and also the addition salts with mineral and organic acids or with mineral and organic bases of said compounds of formula (I).

5) A compound of formula (I) as defined in claim 1, in which R1 is a pyrazolyl or indazolyl radical optionally substituted with one or more radicals selected from halogen atoms and R4, OR4, SR4, thienyl, —N(R5)C(═O)R4, —N(R5)SO2R4, —NY1Y2, —C(═O)NY1Y2 and —NH—C(═O)NY1Y2 radicals,
R2 and R3, which may be identical or different, are selected from hydrogen, halogen, hydroxyl, alkyl, alkoxy, nitro, cyano, phenyl, phenoxy, a carboxyl radical which is free, salified or esterified with an alkyl radical or amidated with NY1Y2 wherein Y1 and Y2, which may be identical or different, are selected fromhydrogen, alkyl, phenyl, phenylalkyl, cycloalkylalkyl, cycloalkyl, furylalkyl and pyridylcarboxyl radicals,
or Y1 and Y2, together with the nitrogen atom to which they are attached, form a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,
R4 is selected from alkyl, cycloalkyl, phenyl and cycloalkylalkyl, which are optionally substituted,
R5 is hydrogen or an optionally substituted alkyl,
all the alkyl, alkoxy, phenyl and phenoxy radicals indicated above being optionally substituted with one or more radicals chosen from halogen atoms and hydroxyl, cyano, alkyl, alkoxy, free, salified or esterified carboxyl, NH2, NHAlk, N(Alk)2, NHSO2Alk, phenylamino, phenylalkylamino, phenyl, morpholino, furyl and pyridyl radicals,
all the alkyl, Alk and alkoxy radicals mentioned above being linear or branched and containing not more than 6 carbon atoms,
the said compounds of formula (I) being in all the possible racemic, enantiomeric and diastereoisomeric isomer forms, and also the addition salts with mineral and organic acids or with mineral and organic bases of said compounds of formula (I).

6) A compound of formula (I) as defined in claim 1, in which R1, R2 and R3 are as defined in claim 1, either Y1 and Y2, which may be identical or different, are independently selected from hydrogen, alkyl, phenyl, phenylalkyl, cycloalkylalkyl, cycloalkyl, furylalkyl and pyridylcarboxyl,
or Y1 and Y2, together with the nitrogen atom to which they are attached, form a pyrrolidinyl, morpholino or piperazinyl radical optionally substituted on the second nitrogen atom with an alkyl or phenyl radical, which are themselves optionally substituted with an NH2, NHAlk, N(Alk)2 or NHSO2Alk radical, or a morpholino, furyl or pyridyl radical,
the said compound of formula (I) being in all the possible racemic, enantiomeric and diastereoisomeric isomer forms, and also the addition salts with mineral and organic acids or with mineral and organic bases of said compounds of formula (I).

7) A compound of formula (I) as defined in claim 1, in which R1 is a pyrazolyl radical optionally substituted with one or two substituents selected from the group consisting of halogen,hydroxyl, nitro, cyano, R4, OR4, SR4, —COR4, —OC(═O)R4, —C(═O)OR4, free —C(═O)OH or a salt thereof, —N(R5)C(═O)R4, —N(R5)C(═O)OR4, —S(O)nR4,
—S(O)nOR4, —N(R5)SO2R4, —OS(O)nR4, —NY1Y2, —C(═O)NY1Y2, —OC(═O)NY1Y2, —N(R5)C(═O)NY1Y2, —S(O)nNY1Y2 and thienyl radicals, which radicals are optionally substituted,
R2, R3, R4 and R5 being as defined in claim 1,
the said compound of formula (I) being in all the possible racemic, enantiomeric and diastereoisomeric isomer forms, and also the addition salts with mineral and organic acids or with mineral and organic bases of the said compound of formula (I).

8) A compound of formula (I) as defined in claim 1, in which R1 is a pyrazolyl radical corresponding to formula (P): 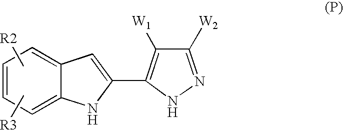
in which R2 and R3 are as defined in claim 1 and W1 and W2 are such that:
either W1 and W2, which may be identical or different, are selected from hydrogen, OR4, SR4, —N(R5)C(═O)R4, —N(R5)SO2R4, —NY1Y2, —N(R5)C(═O)NY1Y2 and —C(═O)NY1Y2, or one of W1 and W2 is hydrogen, OR4 or SR4 and the other is selected from hydrogen, —N(R5)C(═O)R4, —N(R5)SO2R4, —NY1Y2(NH2), —N(R5)C(═O)NY1Y2 and —C(═O)NY1Y2, or W1 is selected from hydrogen, —N(R5)C(═O)R4, —N(R5)SO2R4, —NY1Y2(NH2), —N(R5)C(═O)NY1Y2 or —C(═O)NY1Y2 and W2 represents hydrogen, OR4 and SR4,
it being understood that W1 and W2 are not both hydrogen, and
R4, R5, Y1 and Y2 are as defined in claim 1,
said compound of formula (I) being in all the possible racemic, enantiomeric and diastereoisomeric isomer forms, and also the addition salts with mineral and organic acids or with mineral and organic bases of said compounds of formula (I).

9) A compound of formula (I) as defined in claim 1, in which R1 is a pyrazolyl radical substituted with two substituents W1 and W2 such that one is hydrogen, OR4 or SR4 and the other is hydrogen, —N(R5)C(═O)R4, —N(R5)SO2R4, —NY1Y2, —C(═O)NY1Y2 or —NH—C(═O)NY1Y2,
it being understood that W1 and W2 are not both hydrogen,
R4 is alkyl, cycloalkyl or phenyl, which are optionally substituted,
R5 is hydrogen or an optionally substituted alkyl,
NY1Y2 are such that either Y1 and Y2, which may be identical or different, are selected from hydrogen and optionally substituted alkyl and pyridylcarboxyl radicals, or Y1 and Y2, together with the nitrogen atom to which they are attached, form a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,
all the alkyl, alkoxy and phenyl radicals indicated above also being optionally substituted with an NH2, NHAlk, N(Alk)2 or NHSO2Alk radical or a morpholino, furyl or pyridyl radical,
or a phenyl radical itself optionally substituted with one or more radicals chosen from halogen atoms and alkyl, salified or esterified free carboxyl, amino, alkylamino, dialkylamino, phenylamino, hydroxyl, alkoxy and NHCOalk radicals,
all the alkyl, Alk and alkoxy radicals indicated above being linear or branched and containing not more than 6 carbon atoms,
all the pyridyl radicals themselves being optionally substituted with a halogen atom,
R2 and R3 being as defined in claim 1,
said compound of formula (I) being in all the possible racemic, enantiomeric and diastereoisomeric isomer forms, and also the addition salts with mineral and organic acids or with mineral and organic bases of said compound of formula (I).

10) A compound of formula (I) as defined in claim 1, in which R1 is a pyrazolyl radical substituted with two substituents W1 and W2, one of said W1 and W2 being selected from hydrogen, OR4 and SR4 and the other being selected from hydrogen, —N(R5)C(═O)R4, —N(R5)SO2R4, —NY1Y2, —C(═O)NY1Y2 and —NH—C(═O)NY1Y2,
it being understood that W1 and W2 are not both hydrogen,
R4 is alkyl, cycloalkyl or phenyl, which are optionally substituted,
R5 is hydrogen or an optionally substituted alkyl,
NY1Y2 being such that either Y1 and Y2, which may be identical or different, are selected from hydrogen and optionally substituted alkyl and pyridylcarboxyl radicals, or Y1 and Y2, together with the nitrogen atom to which they are attached, form a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,
all the alkyl, alkoxy and phenyl radicals indicated above also being optionally substituted with an NH2, NHAlk, N(Alk)2 or NHSO2Alk radical or a morpholino, furyl or pyridyl radical,
all the alkyl, Alk and alkoxy radicals indicated above being linear or branched and containing not more than 6 carbon atoms,
all the pyridyl radicals themselves being optionally substituted with a halogen atom,
R2 and R3 being as defined in claim 1,
said compound of formula (I) being in all the possible racemic, enantiomeric and diastereoisomeric isomer forms, and also the addition salts with mineral and organic acids or with mineral and organic bases of said compounds of formula (I).

11) A compound of formula (I) as defined in claim 9, in which R1 is a pyrazolyl radical substituted with two substituents W1 and W2 as defined above, such that one is hydrogen and the other is OR4;
R4 is alkyl, cycloalkyl or phenyl optionally substituted with an NH2, NHAlk, N(Alk)2 or NHSO2Alk radical, a morpholino, furyl or pyridyl radical, or a phenyl radical itself optionally substituted with one or more radicals selected from halogen, amino, alkylamino, dialkylamino, phenylamino, hydroxyl, alkoxy and NHCOalk,
all the alkyl, Alk and alkoxy radicals indicated above being linear or branched and containing not more than 6 carbon atoms,
R2 and R3 being as defined in claim 9,
said compound of formula (I) being in all the possible racemic, enantiomeric and diastereoisomeric isomer forms, and also the addition salts with mineral and organic acids or with mineral and organic bases of said compounds of formula (I).

12) A compound of formula (I) as defined in claim 5, in which R1 is an indazolyl radical optionally substituted with one or more substituents selected from the group consisting of halogen, hydroxyl, nitro, cyano, R4, OR4, SR4, —COR4, —OC(═O)R4, —C(═O)OR4, free —C(═O)OH or a salt thereof, —N(R5)C(═O)R4, —N(R5)C(═O)OR4, —S(O)nR4,
—S(O)nOR4, —N(R5)SO2R4, —OS(O)nR4, —NY1Y2, —C(═O)NY1Y2, —OC(═O)NY1Y2, —N(R5)C(═O)NY1Y2, —S(O)nNY1Y2 and thienyl radicals, which radicals are optionally substituted,
R2, R3, R4 and R5 each being as defined in claim 5,
said compound of formula (I) being in all the possible racemic, enantiomeric and diastereoisomeric isomer forms, and also the addition salts with mineral and organic acids or with mineral and organic bases of said compounds of formula (I).

13) A compound of formula (I) as defined in claim 1, in which R1 is an indazolyl radical,
one of R2 and R3 is hydrogen and the other is selected from the group consisting of hydrogen, halogen, alkyl radicals optionally substituted with a radical selected from NY1Y2, alkoxy, cyano and carboxyl which is free, salified or esterified with an alkyl radical or amidated as a radical CONY1Y2,
NY1Y2 being such that either Y1 and Y2, which may be identical or different, are selected from hydrogen, alkyl and pyridylcarboxyl radicals, or Y1 and Y2, together with the nitrogen atom to which they are attached, form a pyrrolidinyl, pyrazolidinyl, pyrazolinyl, piperidyl or morpholino radical or a piperazinyl radical optionally substituted with an alkyl or phenyl radical, which are themselves optionally substituted,
all the alkyl, alkoxy and phenyl radicals indicated above also being optionally substituted with an NH2, NHAlk, N(Alk)2 or NHSO2Alk radical or a morpholino, furyl or pyridyl radical,
Alk meaning alkyl,
all the alkyl, Alk and alkoxy radicals indicated above being linear or branched and containing not more than 4 carbon atoms,
all the pyridyl radicals themselves being optionally substituted with a halogen atom,
said compound of formula (I) being in all the possible racemic, enantiomeric and diastereoisomeric isomer forms, and also the addition salts with mineral and organic acids or with mineral and organic bases of said compounds of formula (I).

14) A compound of formula (I) as defined in claim 1, in which R1 is an indazolyl radical,
one of R2 and R3 is hydrogen and the other is selected fromhydrogen, halogen, alkyl radicals optionally substituted with NY1NY2, alkoxy radicals optionally substituted with a morpholino radical, a cyano radical or a carboxyl radical which is free, salified or esterified with an alkyl radical or amidated as a radical CONY1Y2,
either Y1 and Y2, which may be identical or different, are selected fromhydrogen, alkyl, furylalkyl, pyridylcarboxyl and pyridylalkyl radicals in which the pyridyl radicals are themselves optionally substituted with a halogen atom, or Y1 and Y2, togetherwith the nitrogen atom to which they are attached, form a piperazinyl radical optionally substituted with an alkyl or phenyl radical, which are themselves optionally substituted with an NHSO2CH3, NH2, NHAlk or N(Alk)2 radical,
all the alkyl or Alk and alkoxy radicals indicated above being linear or branched and containing not more than 4 carbon atoms,
said compound of formula (I) being in all the possible racemic, enantiomeric and diastereoisomeric isomer forms, and also the addition salts with mineral and organic acids or with mineral and organic bases of said compounds of formula (I).

15) A compound of formula (I) as defined in claim 1, selected from the group consisting of:
3-(5-cyanoindol-2-yl)indazole
3-(indol-2-yl)indazole
3-(5-ethoxycarbonylindol-2-yl)indazole
3-(5-(N,N-diisopropyl)carboxamideindol-2-yl)indazole
3-(5-methylindol-2-yl)indazole
3-(5-chloroindol-2-yl)indazole
3-(6-methylindol-2-yl)indazole
3-(5-carboxyindol-2-yl)indazole
3-(5-(N-(2-chloropyridin-5-yl)methyl)carboxamideindol-2-yl)indazole
3-(5-(morpholinoethyloxy)indol-2-yl)indazole
3-(5-aminomethylindol-2-yl)indazole
3-(5-(N-((2-furyl)methyl))carboxamideindol-2-yl)indazole
3-(6-methoxycarbonylindol-2-yl)indazole
3-(5-((2-chloropyrid-5-yl)carboxamido)methylene)indol-2-yl)indazole
3-(6-carboxyindol-2-yl)indazole
3-(6-(N-(2-chloropyrid-5-yl)methyl)carboxamideindol-2-yl)indazole
3-(6-(N-((2-furyl)methyl))carboxamideindol-2-yl)indazole
3-(5-(N-(4-methylsulphonamidephenyl)piperazinocarboxamide)indol-2-yl)indazole
4-amino-3-(indol-2-yl)pyrazole
3-[5-(1H-indol-2-yl)-2H-pyrazol-3-yloxymethyl]phenol
N-{3-[5-(indol-2-yl)-2H-pyrazol-3-yloxymethyl]phenyl}acetamide
2-[5-(3-fluorobenzyloxy)-1H-pyrazol-3-yl]-1H-indole
and also the addition salts with pharmaceutically acceptable mineral and organic acids or with pharmaceutically acceptable mineral and organic bases of said compounds of formula (I).

16) A pharmaceutical composition comprising a therapeutically effective amount of at least one compound of formula (I) as defined in claim 1 or a pharmaceutically acceptable salt of said compound or a prodrug of said compound.

17) A method of inhibiting the activity of a protein kinase comprising administering to a patient in need thereof a therapeutically effective amount of a compound of claim 1.

18) The method of claim 17 which comprises deregulation of the activity of a protein kinase.

19) The method of claim 17 in which the protein kinase is a tyrosine kinase protein.

20) The method of claim 19, in which the protein kinase is selected from FGFR1, FGFR2, FGFR3, FGFR4, FGFR5, flt-1, IGF-1R, KDR, PDGFR, tie2 and VEGFR.

21) The method of claim 20, in which the protein kinase is KDR.

22) The method of claim 20 in which the protein kinase is tie2.

23) The method of claim 17, in which the protein kinase is in a cell culture.

24) The method of claim 17, in which the protein kinase is in a mammal.

25) A method for treating or preventing a disease selected from the group consisting of disorders of the proliferation of blood vessels, fibrotic disorders, disorders of the proliferation of “mesangial” cells, metabolic disorders, allergies, asthma, thrombosis, diseases of the nervous system, retinopathy, psoriasis, rheumatoid arthritis, diabetes, muscle degeneration and cancers comprising administering to a patient in need thereof an effective amount of a compound of claim 1.

26) The method of claim 25 wherein the disease is selected from disorders of the proliferation of blood vessels, fibrotic disorders, disorders of the proliferation of “mesangial” cells, retinopathy, psoriasis, rheumatoid arthritis, diabetes, muscle degeneration and cancers.

27) The method of claim 25 wherein said disease is associated with uncontrolled angiogenesis.

28) The method of claim 25 wherein said disease is an oncology disease.

29) The method of claim 28 wherein said disease is a cancer.

30) The method of claim 29, wherein said cancer is a solid tumor.

31) The method of claim 29 wherein said cancer is resistant to cytotoxic agents.

32) The method of claim 29 wherein said cancer is selected from breast cancer, stomach cancer, cancer of the ovaries, cancer of the colon, lung cancer, brain cancer, cancer of the larynx, cancer of the lymphatic system, cancer of the genito-urinary tract including the bladder and the prostate, bone cancer and cancer of the pancreas.
